(19) United States
(12) Patent Application Publication
(10) Pub. No.: US 2009/0281340 A1
(43) Pub. Date: Nov. 12, 2009

(54) 1ALPHA-HYDROXY-2-(3'-HYDROXYPROPYLIDENE)-19-NOR-VITAMIN D COMPOUNDS AND METHODS OF MAKING AND TREATMENT THEREOF

(76) Inventors: Hector F. DeLuca, Deerfield, WI (US); Rafal R. Sicinski, Warsaw (PL); Lori A. Plum, Arena, WI (US); Margaret Clagett-Dame, Deerfield, WI (US); Agnieszka Glebocka, Warsaw (PL)

Correspondence Address: QUARLES & BRADY LLP, 411 E. WISCONSIN AVENUE, SUITE 2040, MILWAUKEE, WI, 53202-4497, US

(21) Appl. No.: 12/437,892

(22) Filed: May 8, 2009

Related U.S. Application Data
(63) Continuation-in-part of application No. 11/732,924, filed on Apr. 5, 2007.
(60) Provisional application No. 60/789,303, filed on Apr. 5, 2006.
(60) Provisional application No. 60/744,383, filed on Apr. 6, 2006.
(60) Provisional application No. 60/744,385, filed on Apr. 6, 2006.
(60) Provisional application No. 60/744,379, filed on Apr. 6, 2006.
(60) Provisional application No. 60/744,381, filed on Apr. 6, 2006.
(60) Provisional application No. 60/744,386, filed on Apr. 6, 2006.
(60) Provisional application No. 60/791,487, filed on Apr. 10, 2006.
(60) Provisional application No. 60/791,227, filed on Apr. 6, 2006.

(51) Int. Cl. C07C 401/00; C07C 45/00

(52) U.S. Cl. 552/653; 568/346

(57) ABSTRACT

Disclosed are 1α-hydroxy-2-(3′-hydroxypropylidene)-19-nor-vitamin D compounds, pharmaceutical compositions, and methods of making and treatment thereof. The compounds are generally directed to biologically active 2-alkylidene-19-nor-vitamin D compounds and analogs thereof characterized by the presence of a 3′-hydroxypropylidene moiety at C-2 and the presence of an abbreviated alkyl side-chain free of any hydroxyl moiety.

### CROSS-REFERENCE TO RELATED APPLICATIONS

This application is a continuation-in-part of U.S. Non-Provisional application Ser. No. 11/732,924 filed Apr. 5, 2007. The application is incorporated herein by reference in its entirety.

This application claims the benefit of U.S. Provisional Application Ser. No. 60/789,303 filed Apr. 5, 2006. The application is incorporated herein by reference in its entirety.

This application is related to U.S. Provisional Application Ser. Nos. 60/744,383; 60/744,385; 60/744,379; 60/744,381, 60/744,386; 60/791,487, and 60/791,227.

### FIELD OF THE INVENTION

The instant invention relates to the field of Vitamin D analog compounds and methods of making and treatment thereof.

### BACKGROUND OF THE INVENTION

The natural hormone, 1α,25-dihydroxyvitamin D₃ and its analog in the ergosterol series (i.e., 1α,25-dihydroxyvitamin D₂) are potent regulators of calcium homeostasis in animals and humans. Recently, its cellular differentiation activity has been established, see Ostrem et al., Proc. Natl. Acad. Sci. USA, 84, 2610 (1987). Structural analogs of these metabolites have been prepared and tested such as 1α-hydroxyvitamin D₃, 1α-hydroxyvitamin D₂, and, various side-chain homologated vitamins and fluorinated analogs thereof. Some of these compounds exhibit separation of activities in cell differentiation and calcium regulation. The difference in activity may be advantageous in treating a variety of diseases such as renal osteodystrophy, vitamin D-resistant rickets, osteoporosis, psoriasis, and other malignancies.

A class of vitamin D analogs, the 19-nor-vitamin D compounds, are characterized by replacement of the A-ring exocyclic methylene group at the carbon 19 (typical of the vitamin D system) with two hydrogen atoms. Biological testing of such 19-nor-analogs (e.g., 1α,25-dihydroxy-19-nor-vitamin D₃) revealed a selective activity profile having high potency to induce cellular differentiation and very low calcium mobilizing activity. Potentially, these compounds are useful therapeutic agents for treating renal osteodystrophy, vitamin D-resistant rickets, osteoporosis, psoriasis, other malignancies and various skin disorders.

Two different synthetic methods of making various 19-nor-vitamin D analogs have been described—See Perlman et al., Tetrahedron Letters 31, 1823 (1990); Perlman et al. Tetrahedron Letters 32, 7663 (1991); and, DeLuca et al., U.S. Pat. No. 5,086,191. Analogs of 1α,25-dihydroxy-19-norvitamin D₃ substituted at 2-position with hydroxy or alkoxy groups have also been synthesized (see DeLuca et al., U.S. Pat. No. 5,536,713) which may exhibit selective activity profiles.

Analogs characterized by the transposition of the A-ring exocyclic methylene group from carbon 10 (C10) to carbon 2 (C2) (e.g., 2-methylene-19-nor-vitamin D compounds) have been synthesized and tested. (See Sicinski et al., J. Med. Chem., 41, 4662 (1998); Sicinski et al., Steroids 67, 247 (2002); and, DeLuca et al., U.S. Pat. Nos. 5,843,928; 5,936,133 and 6,382,071). Molecular mechanics studies performed on these analogs predict that a change of A-ring conformation may cause flattening of the cyclohexanediol ring. Molecular mechanics calculations and NMR studies also predict that the A-ring conformational equilibrium would be ca. 6:4 in favor of the conformer having an equatorial 1α-OH. It was further predicted that introduction of the 2-methylene group into 19-nor-vitamin D carbon skeleton would change the character of its 1α- and 3β-A-ring hydroxyls. They would both be in allylic positions similar to the 1α-hydroxyl group (which is important for biological activity) in the molecule of the natural hormone (i.e., 1α,25-(OH)₂D₃). It was found that 1α,25-dihydroxy-2-methylene-19-norvitamin D analogs are characterized by significant biological potency. In addition, the biological potency of such analogs may be enhanced dramatically where “unnatural” (20S) configuration is present.

Recently, 2-ethylidene analogs of 1α,25-dihydroxy-19-norvitamin D₃ have been synthesized whereby such modification of the A-ring resulted in significant biological potency particularly for the E-geometrical isomers, see Sicinski et al., J. Med. Chem., 45, 3366 (2002). It has been established that E-isomers have A-ring conformational equilibrium that is considerably shifted to the chair form possessing 1α-hydroxyl in equatorial orientation.

Recently, derivatives of 1α,25-dihydroxy-19-norvitamin D₃ having a 3′-hydroxypropylidene moiety at C-2 have been synthesized (see DeLuca et. al, U.S. Patent Application No. 2004/0229851) whereby the in vivo calcemic activity significantly exceeded that of 1α,25-(OH)₂D₃ particularly regarding stimulation of intestinal calcium transport. Molecular modeling studies of the analogs predicted that presence of an oxygen function (located at the terminus of the propylidene fragment) may promote interaction with the vitamin D receptor. The modeling further predicted that affinity of the synthesized compounds to VDR may approach that of the natural hormone. Taking into account the recent findings on 2-methylene-1α-hydroxy-19-norvitamin D analogs having truncated side-chains, Plum et al., PNAS, 101, 6900 (2004), indicates that such compounds effectively suppress parathyroid hormone levels.

### SUMMARY OF THE INVENTION

One aspect of the invention is a compound of Formula I comprising:

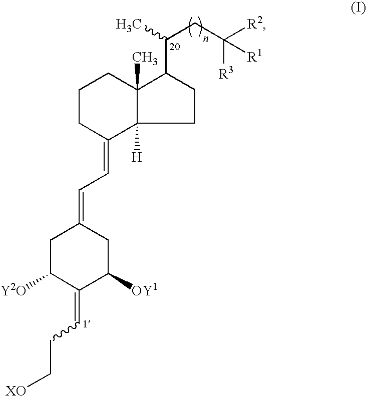

wherein [symbol] at C1′ provides that the compound is an E- or Z-geometrical isomer respecting the 2-propylidene segment, wherein the C20 is the stereochemical center, wherein the [symbol] at C20 provides an R or S configuration, wherein n is an integer from 1 to 3, wherein Y¹ is hydrogen, deuterium or a first hydroxy-protecting group, wherein Y² is hydrogen, deuterium or a second hydroxy-protecting group, wherein X is a third hydroxy-protecting group, wherein R¹ is hydrogen, deuterium or methyl, wherein R² is hydrogen, deuterium or methyl, wherein R³ is hydrogen, deuterium or methyl, and, wherein [symbol] is [symbol] or [symbol] and hydrates thereof.

In another embodiment, X is hydrogen, deuterium, C₁₋₁₀ branched or straight alkyl, C₁₋₁₀ branched or straight alkyl substituted with one or more hydroxy groups, C₁₋₁₀ branched or straight alkyl substituted with one or more C₁₋₁₀ branched or straight alkoxy groups, C₁₋₁₀ branched or straight alkyl substituted with one or more aryloxy groups, carbonyl substituted with one or more C₁₋₁₀ branched or straight alkoxy group, C₁₋₆ branched or straight alkanoyl, C₁₋₆ branched or straight carboxyalkanoyl, aromatic acyl, silyl substituted with one or more C₁₋₁₀ branched or straight alkyl groups, silyl substituted with one or more C₁₋₁₀ branched or straight alkyl groups, or, silyl substituted with one or more aryl groups.

In another embodiment, the carbonyl substituted with a C₁₋₁₀ branched or straight alkoxy group is methoxycarbonyl, ethoxycarbonyl, propoxycarbonyl, iso-propoxycarbonyl, butoxycarbonyl, isobutoxycarbonyl, tert-butoxycarbonyl, benzyloxycarbonyl or allyloxycarbonyl.

In another embodiment, the C₁₋₆ branched or straight carboxyalkanoyl is oxalyl, malonyl, succinyl, or, glutaryl.

In another embodiment, the aromatic acyl is benzoyl, halo-substituted benzoyl, nitro-substituted benzoyl, and, C₁₋₁₀ straight or branched alkyl-substituted benzoyl.

In another embodiment, the C₁₋₁₀ branched or straight alkyl substituted with one or more C₁₋₁₀ branched or straight alkoxy groups is methoxymethyl, ethoxymethyl, methoxyethoxymethyl, tetrahydrofuranyl or tetrahydropyranyl.

In another embodiment, the silyl substituted with one or more C₁₋₁₀ branched or straight alkyl groups is trimethylsilyl, triethylsilyl, t-butyldimethylsilyl, and, dibutylmethylsilyl.

In another embodiment, the silyl substituted with one or more aryl groups is diphenylmethylsilyl, phenyldimethylsilyl, or diphenyl-t-butylsilyl.

In another embodiment, the C₁₋₁₀ branched or straight alkyl substituted with one or more aryloxy groups is phenyl-substituted phenyl, C₁₋₁₀ straight or branched alkyl-substituted phenyl, nitro-substituted phenyl, or halo-substituted phenyl.

In another embodiment, the compound is an E-geometrical isomer. Alternatively, the compound is a Z-geometrical isomer.

In another embodiment, X is t-butyldimethylsilyl, Y¹ is t-butyldimethylsilyl, and Y² is t-butyldimethylsilyl. Alternatively, X is hydrogen, Y¹ is hydrogen, and Y² is hydrogen.

In another embodiment, n is 1, R¹ and R² are methyl, and R³ is hydrogen.

In another embodiment, the compound is an E-isomer of (20R)-1α-hydroxy-2-(3′-hydroxypropylidene)-19,24,25,26,27-penta-nor-vitamin D₃.

In another embodiment, the compound is an E-isomer of (20S)-1α-hydroxy-2-(3′-hydroxypropylidene)-19,24,25,26,27-penta-nor-vitamin D₃.

In another embodiment, the compound is an E-isomer of (20R)-1α-hydroxy-2-(3′-hydroxypropylidene)-19,23,24-tri-nor-vitamin D₃.

In another embodiment, the compound is an E-isomer of (20S)-1α-hydroxy-2-(3′-hydroxypropylidene)-19,23,24-tri-nor-vitamin D₃.

Another aspect of the invention includes a method of making a hydrindanone intermediate compound for use in making the compound of Formula I, wherein n is 1, wherein R¹, R², and R³ are each hydrogen, and, wherein [symbol] at C20 is [symbol] comprising the steps of: providing a starting compound of the Formula II,

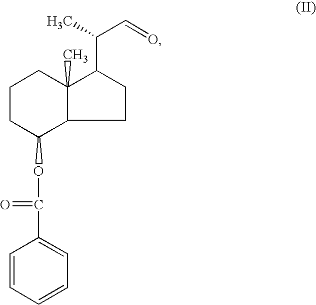

reacting the starting compound with a ylide reactant to produce an alkene-containing product, hydrogenating the alkene-containing product to produce an oily ester product, hydrolysing the oily ester product to produce an alcohol product, and, oxidizing the alcohol product to produce the hydrindanone intermediate compound having the Formula III,

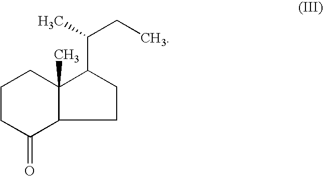

Another aspect of the invention includes a method of making the compound of Formula I, wherein n is 1, wherein R¹, R², and R³ are each hydrogen, and, wherein [symbol] at C20 is [symbol] comprising the steps of coupling the hydrindanone intermediate compound of Formula III with lithium phosphinoxy carbanion to produce a coupled product having the protecting groups, and, hydrolyzing the protecting groups.

Another aspect of the invention includes a method of making a hydrindanone intermediate compound for use in making the compound Formula I, wherein n is 1, wherein R¹, R², and R³ are each hydrogen, and, wherein [symbol] at C20 is [symbol] comprising the steps of providing a starting compound of the Formula IV,

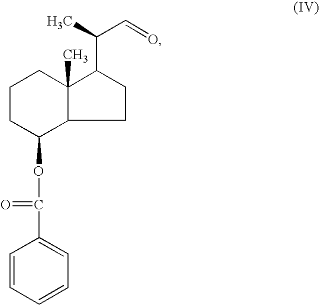

reacting the starting compound with a ylide reactant to produce an alkene-containing product, hydrogenating the alkene-containing product to produce an oily ester product, hydrolysing the oily ester product to produce an alcohol product, and, oxidizing the alcohol product to produce the hydrindanone intermediate compound having the Formula V,

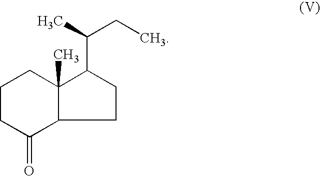

Another aspect of the invention includes a method of making the compound of Formula I, wherein n is 1, wherein R¹, R², and R³ are each hydrogen, and, wherein [symbol] at C20 is [symbol] comprising the steps of coupling the hydrindanone intermediate compound of Formula V with lithium phosphinoxy carbanion to produce a coupled product having the protecting groups, and, hydrolyzing the protecting groups.

Another aspect of the invention includes a method of making the compound of Formula I, wherein at least one of R¹, R² or R³ is a methyl, and, wherein [symbol] at C20 is [symbol] comprising the steps of providing a starting compound of the Formula VI,

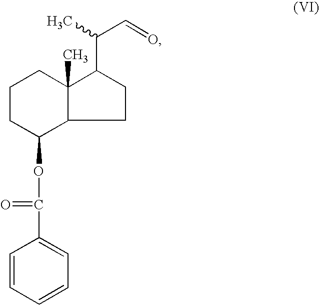

wherein [symbol] is [symbol] or [symbol] converting the starting compound into a nitrile compound, alkylating the nitrile compound with a first reactant of the Formula VII,

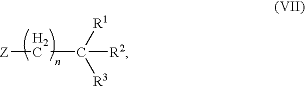

wherein n is an integer from 1 to 3, wherein Z is Br, Cl or I, and, wherein at least one of R¹, R² or R³ is a methyl to produce an alkylated nitrile product, hydrolysing the alkylated nitrile product to produce a hydroxy nitrile product, reductively decyanating the hydroxy nitrile product to produce a mixture of epimeric alcohol products, oxidizing the mixture of epimeric alcohol products to produce a mixture of a 20S-ketone product and a 20R-ketone product, separating the 20S-ketone and 20R-ketone products, coupling the 20R-ketone product with lithium phosphinoxy carbanion to produce a coupled 20R product having the protecting groups, and, hydrolysing the protecting groups. In another embodiment of the method, n is 1, Z is Br, R¹ and R² are methyl, and, R³ is hydrogen.

Another aspect of the invention is a method of making the compound of Formula I, wherein at least one of R¹, R² or R³ is a methyl, and, wherein [symbol] at C20 is [symbol] comprising the steps of providing a starting compound of the Formula VIII,

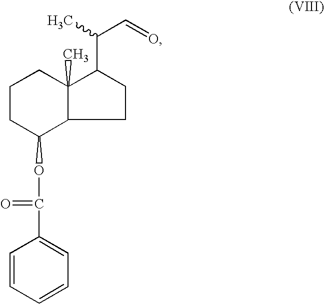

wherein [symbol] is [symbol] or [symbol] converting the starting compound into a nitrile compound, alkylating the nitrile compound with a first reactant of the Formula VII,

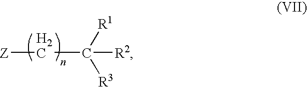

wherein n is an integer from 1 to 3, wherein Z is Br, Cl or I, and, wherein at least one of R¹, R² or R³ is a methyl, to produce an alkylated nitrile product, hydrolysing the alkylated nitrile product to produce a hydroxy nitrile product, reductively decyanating the hydroxy nitrile product to produce a mixture of epimeric alcohol products, oxidizing the mixture of epimeric alcohol products to produce a mixture of a 20S-ketone product and a 20R-ketone product, separating the 20S-ketone and 20R-ketone products, coupling the 20S-ketone product with lithium phosphinoxy carbanion to produce a coupled 20S product having the protecting groups, and, hydrolysing the protecting groups. In another embodiment of the method, n is 1, Z is Br, R¹ and R² are methyl, and, R³ is hydrogen.

### BRIEF DESCRIPTION OF THE EXEMPLARY DRAWINGS

FIG. 1 is a graph illustrating the relative activity of (20S)-1α-hydroxy-2-(3′-hydroxypropylidene)-19,23,24-tri-nor-vitamin D₃ (“HPBS”) and (20R)-1α-hydroxy-2-(3′-hydroxypropylidene)-19,23,24-tri-nor-vitamin D₃ (“HPBR”) as against 1α,25-dihydroxyvitamin D₃ in terms of competitive VDR binding (i.e., binding to the 1α,25-dihydroxyvitamin D₃ pig intestinal nuclear receptor), whereby the procedure set forth in Dame et al (Biochemistry 25, 4523-4534 (1986)) was followed.

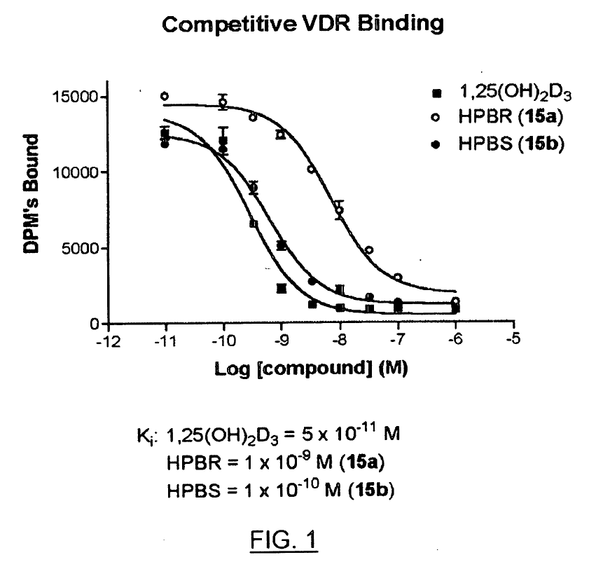

FIG. 2 is a graph illustrating the relative activity of (20R)-1α-hydroxy-2-(3′-hydroxypropylidene)-19,24,25,26,27-penta-nor-vitamin D₃ (“RBH”) and (20S)-1α-hydroxy-2-(3′-hydroxypropylidene)-19,23,24-tri-nor-vitamin D₃ (“SBH”) as against 1α,25-dihydroxyvitamin D₃ in terms of competitive VDR binding (i.e., binding to the 1α,25-dihydroxyvitamin D₃ pig intestinal nuclear receptor), whereby the procedure set forth in Dame et al (Biochemistry 25, 4523-4534 (1986)) was followed.

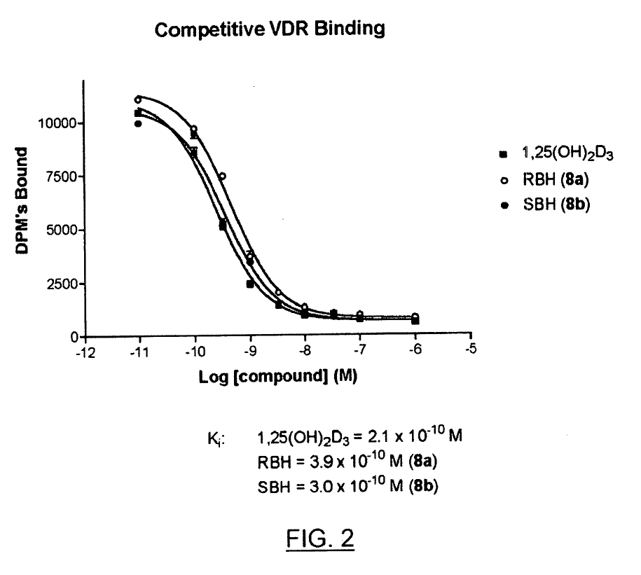

FIG. 3 is a graph illustrating the percent HL-60 cell differentiation activity of 1α,25-dihydroxyvitamin D₃, RBH, and SBH as a function of concentration in the medium, whereby the differentiation of HL-60 promyelocytic into monocytes was determined as set forth in Ostrem et al (J. Biol. Chem. 262, 14164-14171 (1987)).

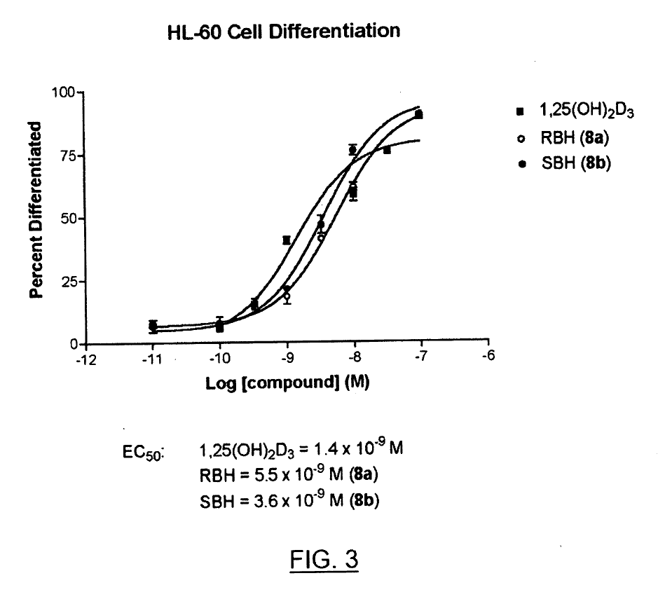

FIG. 4 is a graph illustrating the percent HL-60 cell differentiation activity of 1α,25-dihydroxyvitamin D₃, HPBS, and HPBR as a function of concentration in the medium, whereby the differentiation of HL-60 promyelocytic into monocytes was determined as set forth in Ostrem et al (J. Biol. Chem. 262, 14164-14171 (1987)).

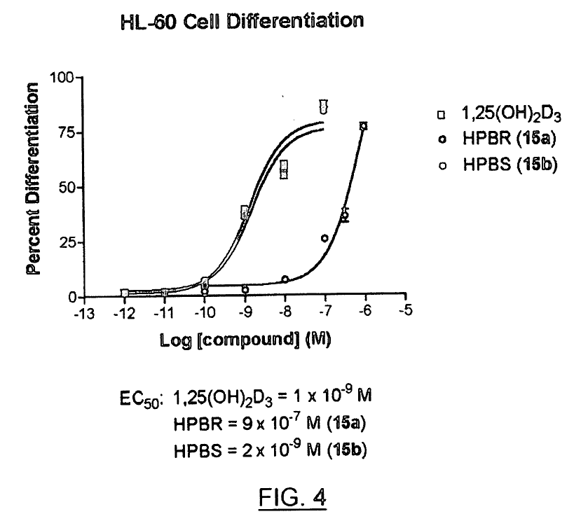

FIG. 5 is a graph illustrating the transcriptional activity of 1α,25-dihydroxyvitamin D₃, RBH, and SBH as a function of concentration, whereby transcriptional activity was measured in ROS 17/2.8 (bone) cells that were stably transfected with a 24-hydroxylase (“24OHase”) gene promoter upstream of a luciferase reporter gene (see Arbour et al, (1998); and Arbour et al, Nat. Genet. 25, 187 (2000)), whereby cells were given a range of doses, whereby cells were harvested 16 hours after dosing, and the luciferase activities were measured using a luminometer, and whereby “RLU” refers to relative luciferase units.

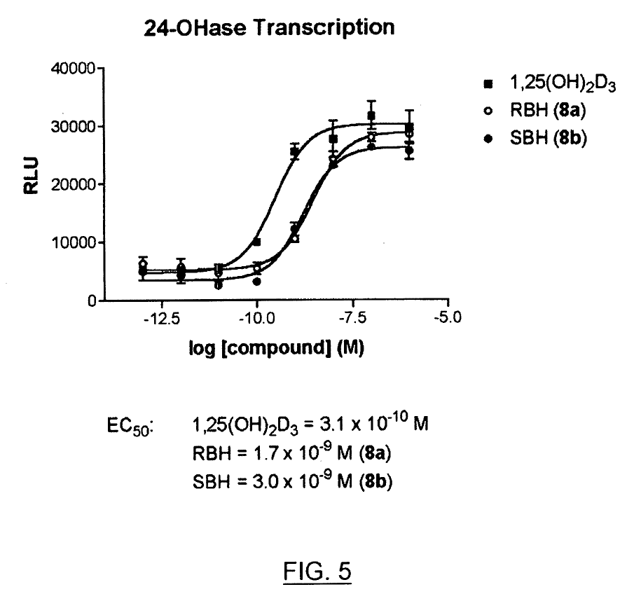

FIG. 6 is a graph illustrating the transcriptional activity of 1α,25-dihydroxyvitamin D₃, HPBR, and HPBS as a function of concentration, whereby transcriptional activity was measured in ROS 17/2.8 (bone) cells that were stably transfected with a 24-hydroxylase (“24OHase”) gene promoter upstream of a luciferase reporter gene (see Arbour et al, (1998); and Arbour et al (2000)), whereby cells were given a range of doses, whereby cells were harvested 16 hours after dosing, and the luciferase activities were measured using a luminometer, and whereby “RLU” refers to relative luciferase units.

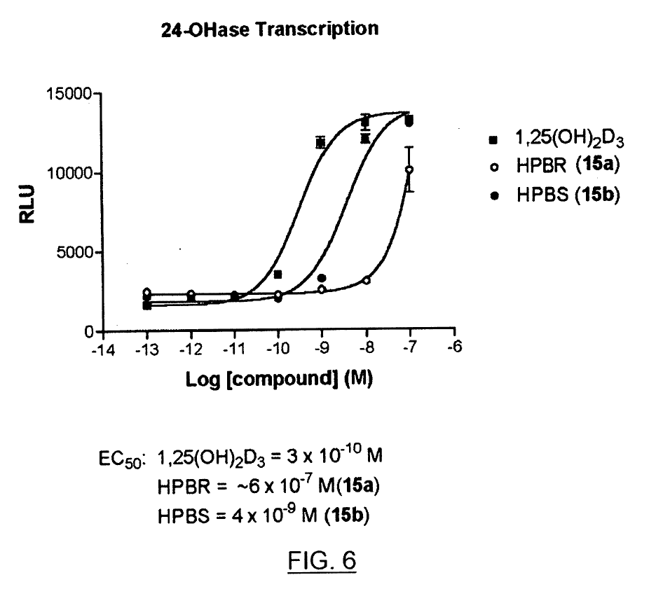

FIG. 7 is bar graphs illustrating bone calcium mobilization activity of 1α,25-dihydroxyvitamin D₃, HPBR, and HPBS administered at various doses to vitamin D deficient rats on a low calcium diet, whereby the rise in serum calcium concentration reflects the mobilization of bone calcium.

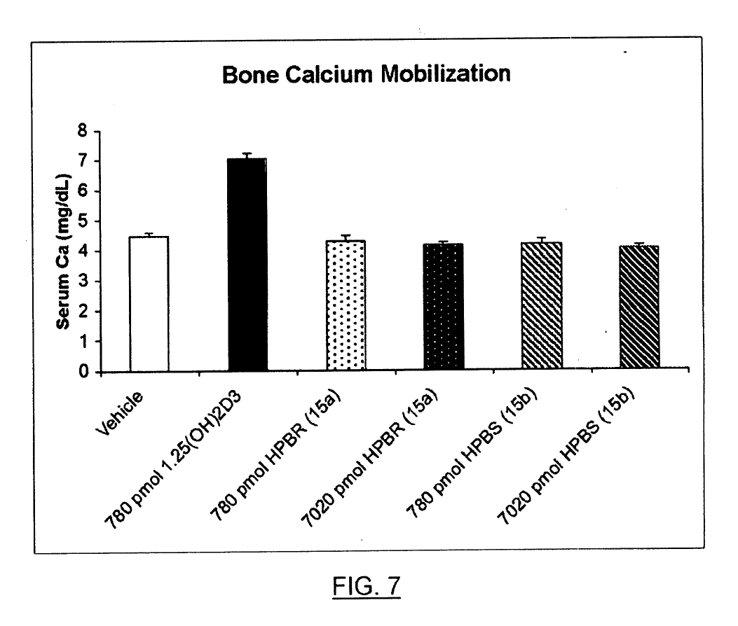

FIG. 8 is a bar graph illustrating bone calcium mobilization activity of the vehicle (i.e., control), 1α,25-dihydroxyvitamin D₃, RBH and SBH administered at various doses to vitamin D deficient rats on a low calcium diet, whereby the rise in serum calcium concentration reflects the mobilization of bone calcium.

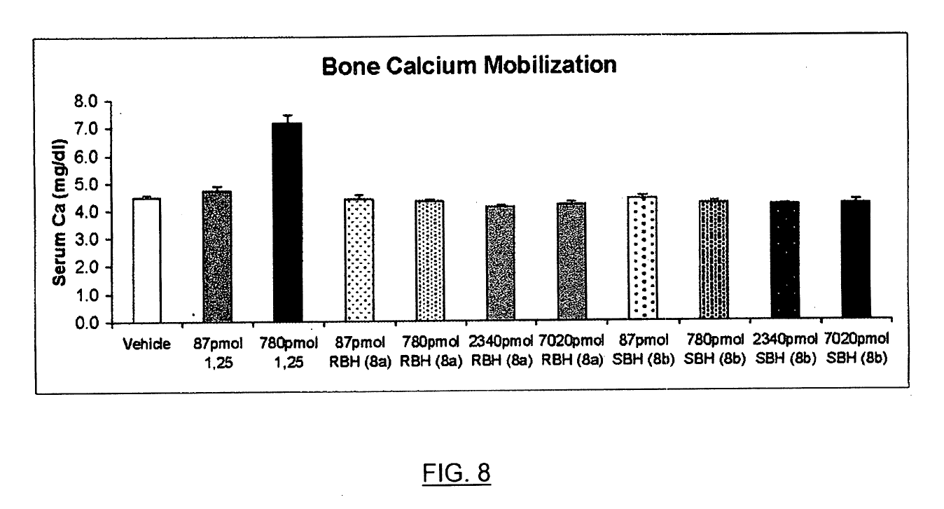

FIG. 9 is a bar graph illustrating intestinal calcium transport activity of the vehicle, 1α,25-dihydroxyvitamin D₃, HPBR, and HPBS administered at various doses to vitamin D deficient rats on a low calcium diet, whereby the intestinal calcium transport was measured by the everted intestinal gut sac method.

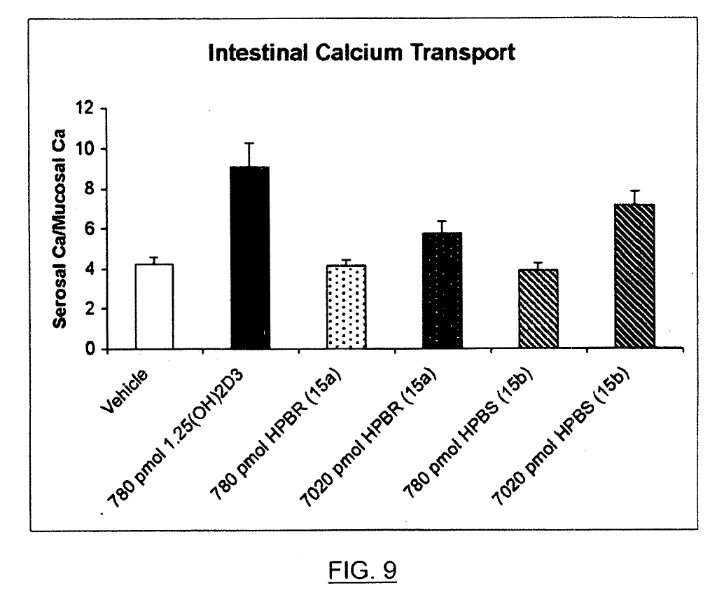

FIG. 10 is a bar graph illustrating intestinal calcium transport activity of the vehicle, 1α,25-dihydroxyvitamin D₃, RBH, and SBH administered at various doses to vitamin D deficient rats on a low calcium diet, whereby the intestinal calcium transport was measured by the everted intestinal gut sac method.

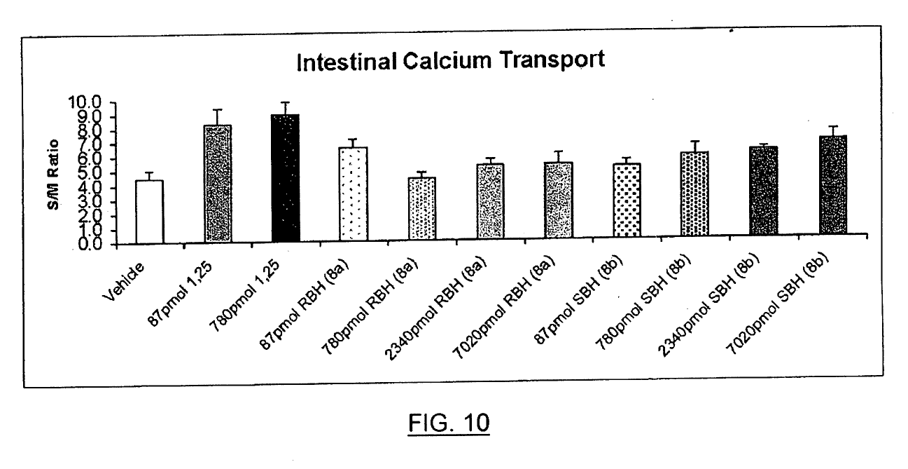

### DETAILED DESCRIPTION OF EXEMPLARY EMBODIMENTS

The instant invention is generally directed to biologically active 2-alkylidene-19-norvitamin D compounds and analogs thereof characterized by the presence of a 3′-hydroxypropylidene moiety at C2 and the presence of an abbreviated alkyl side-chain free of any hydroxyl moiety.

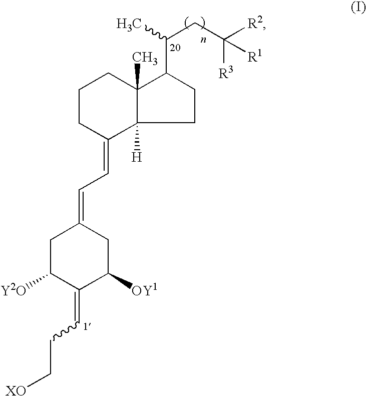

wherein Y₁ and Y₂ (which may be the same or different) is hydrogen or a hydroxy-protecting group; wherein X is alkyl, hydrogen, hydroxy-protecting group, hydroxyalkyl, alkoxyalkyl or aryloxyalkyl, wherein R₁, R₂ and R₃ (which may be the same or different) are hydrogen or methyl, and, wherein [symbol] at C1′ is [symbol] or [symbol] The wavy line attached to C20 indicates that the compound is in either the R or S configuration. The relative position of the 2-propylidene unit at the C1′ indicates the E,Z geometrical isomer configuration relative to the remainder of the molecule.

Exemplary side-chains having a natural 20R- and “unnatural” 20S-configuration includes the structures represented by formulas (a), (b), (c) and (d) below. That is, the side-chain in 24,25,26,27-tetranorvitamin D₃ (also referred to as “bishomopregnacalciferol” and RBH herein) (a); 20S-24,25,26,27-tetranorvitamin D₃ (also referred to as “20S-bishomopregnacalciferol”and SBH herein) (b); 23,24-dinorvitamin D₃ (also referred to as HPBR herein) (c); and, 20S-23,24-dinorvitamin D₃ (also referred to as HPBS herein) (d)

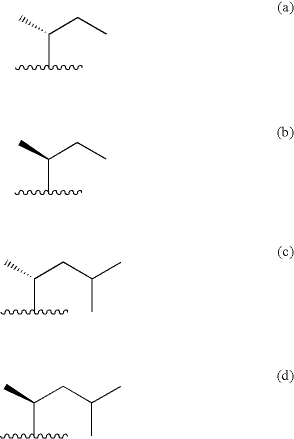

Preparation of 1α-hydroxy-19-nor-vitamin D compounds having the substituted propylidene moiety at C2, of the basic structure I can be accomplished by the condensation of a bicyclic Windaus-Grundmann type ketone II with the allylic phosphine oxide III as set forth below.

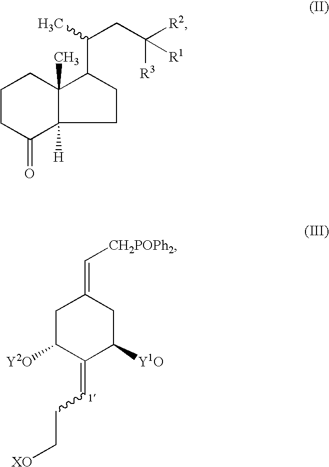

wherein [symbol] at 1′ is [symbol] or

[symbol]

Regarding Formulas II and III, groups Y₁, Y₂, X, R₁, R₂ and R₃ represent groups defined hereinabove; and, preferably, Y₁, Y₂, X are hydroxy-protecting groups. The process is an application of the convergent synthesis concept which has been used to prepare vitamin D compounds. (See, e.g., Lythgoe et al., J. Chem. Soc. Perkin Trans. I, 590 (1978), Lythgoe, Chem. Soc. Rev. 9, 449 (1983), Toh et al., J. Org. Chem. 48, 1414 (1983), Baggiolini et al., J. Org. Chem. 51, 3098 (1986); Sardina et al., J. Org. Chem. 51, 1264 (1986); J. Org. Chem. 51, 1269 (1986); DeLuca et al., U.S. Pat. No. 5,086,191; and, DeLuca et al., U.S. Pat. No. 5,536,713).

The phosphine oxides of III are available or can be prepared from commercially available (1R,3R,4S,5R)-(−)-quinic acid. (See Glebocka et al., J. Steroid Biochem. Mol. Biol. 89-90, 25 (2004); and, DeLuca et. al, US Patent Application No. 2004/0229851).

Regarding preparation of the hydrindanones of II, alternative synthetic routes start from the epimeric at C20 and the 22-aldehydes of 1a and 1b. (See Fall et al., Tetrahedron Lett. 43, 1433 (2002); Granja et al., J. Org. Chem. 58, 124 (1993)). As set forth in SCHEME I, separate analogous processes transform starting aldehydes 1a and 1b into C,D-ring synthons 5a,b that are subsequently coupled with phosphine oxide 6. Aldehydes 1a and 1b were reacted with a ylide generated from methyltriphenylphosphonium bromide and n-butyllithium (i.e., a Wittig reaction). The resulting olefins 2a and 2b were hydrogenated generating saturated compounds 3a and 3b possessing side chains having 4 carbons. Basic hydrolysis of the ester group produced the 8β-alcohols 4a and 4b that were subsequently oxidized with tetrapropylammonium perruthenate to make the hydrindanones 5a and 5b. Wittig-Horner coupling of the Grundmann ketones 5a and 5b with lithium phosphinoxy carbanion, generated from the phosphine oxide (6) (prepared in accordance with DeLuca et. al, U.S. Patent Application No. 2004/0229851, which is incorporated herein by reference) provided the protected vitamin compounds 7a and 7b. After deprotecting with tetrabutyl ammonium fluoride, 1α-hydroxy-2-[3′-hydroxypropylidene]-19,24,25,26,27-pentanorvitamin D₃ compounds 8a and 8b were made. 1α-hydroxy-2-[3′-hydroxypropylidene]-19,24,25,26,27-pentanorvitamin D₃ (8a) is described in EXAMPLE I herein and preparation of its 20S-epimer 8b is in EXAMPLE II herein.

SCHEME II shows preparation of the vitamin D analogs having iso-branched alkyl substituents (i.e., iso-butyl) attached to C20 and starting from the same 22-aldehyde 1a. Aldehyde 1a was transformed into a mixture of isomeric E- and Z-oximes which (upon heating with acetic anhydride) formed the nitrile 9. The nitrile 9 was treated with LDA producing carbanion alkylated by addition of iso-butyl bromide. X-Ray analysis showed that the single alkylation product (10) possessed 20S-configuration.

Subsequently, alkaline hydrolysis of 8β-benzoyloxy group in the nitrile 10 produced the corresponding alcohol 11 which is desirable for reductive removal of the C20 cyano group, whereby the conditions required for such decyanation process could otherwise cause the reduction of the 8-benzoyloxy group to the corresponding alkane (8-unsubstituted derivative). 8β-Hydroxy group in alcohol 11 could be protected as alkylsilyl-, arylsilyl or alkoxyalkyl ether before the decyanation process. Several methods for the reductive decyanation of alcohol 11 are available, whereby dissolving metal reductions are preferred.

For example, alcohol 11 can be transformed into a mixture of alcohols 12a and 12b by reacting with potassium metal in hexamethylphosphoric triamide and tert-butanol or by reacting with a potassium metal/dicyclohexano-18-crown-6/toluene system. 8β-Alcohols 12a and 12b were subsequently oxidized with tetrapropylammonium perruthenate to make the hydrindanones 13a and 13b. Separation of the Grundmann ketones (epimeric at C20) was achieved using HPLC. Wittig-Horner coupling of the hydrindanones 13a and 13b was performed using lithium phosphinoxy carbanion generated from the phosphine oxide 6 and phenyllithium producing protected vitamin compounds 14a and 14b. After de-protecting with tetrabutylammonium fluoride, 1α-hydroxy-2-[3′-hydroxypropylidene]-19,23,24-trinorvitamin D₃ compounds (15a,b) was produced.

As set forth in EXAMPLE III, synthesis of 1α-hydroxy-2-[3′-hydroxypropylidene]-19,23,24-trinorvitamin D₃ (15a) and its epimer 15b is shown. It is appreciated that other 1α-hydroxy-2-[3′-hydroxypropylidene]-19-nor-vitamin D analogs having the instant alkyl side-chains may be synthesized by the methods set forth herein.

This invention is described by the following illustrative examples. In these examples specific products identified by Arabic numerals (e.g. 1, 2, 3, etc) refer to the specific structures identified in the preceding description and in the SCHEME I and SCHEME II.

### EXAMPLES

Chemistry. Melting points (uncorrected) were determined using a Thomas-Hoover capillary melting-point apparatus. Ultraviolet (UV) absorption spectra were recorded using a Perkin-Elmer Lambda 3B UV-VIS spectrophotometer in ethanol. ¹H nuclear magnetic resonance (NMR) spectra were recorded at 400 MHz using a Bruker Instruments DMX-400 Avance console spectrometer in deteriochloroform. Chemical shifts (δ) were determined downfield from internal Me₄Si (δ 0.00). Electron impact (EI) mass spectra were determined using a Micromass AutoSpec (Beverly, Mass.) instrument. High-performance liquid chromatography (HPLC) was determined using a Waters Associates liquid chromatograph equipped with a Model 6000A solvent delivery system, a Model U6K Universal injector, and, a Model 486 tunable absorbance detector. THF was freshly distilled before use from sodium benzophenone ketyl under argon.

Biological Activity; Vitamin D Receptor Binding; Test Material and Protein Source. Full-length recombinant rat receptor was expressed in E. coli BL21 (DE3) Codon Plus RIL cells and purified to homogeneity using two different column chromatography systems. The first system was a nickel affinity resin that utilized the C-terminal histidine tag on the protein. The protein eluted from the resin was further purified using ion exchange chromatography (S-Sepharose Fast Flow). Aliquots of the purified protein were quick frozen in liquid nitrogen and stored at −80° C. until use. For use in binding assays, the protein was diluted in TEDK₅₀ (50 mM Tris, 1.5 mM EDTA, pH=7.4, 5 mM DTT, 150 mM KCl) with 0.1% Chaps detergent. The receptor protein and ligand concentration was optimized such that no more than 20% of the added radiolabeled ligand was bound to the receptor.

Unlabeled ligands were dissolved in ethanol, and the concentrations were determined using UV spectrophotometry (1,25(OH)₂D₃: molar extinction coefficient=18,200 and λₘₐₓ=265 nm; Analogs: molar extinction coefficient=42,000 and λₘₐₓ=252 nm).

Radiolabeled ligand (³H-1,25(OH)₂D₃) was added in ethanol at a final concentration of 1 nM.

Radiolabeled and unlabeled ligands were added to 100 mcl of the diluted protein at a final ethanol concentration of <10%, mixed and incubated overnight on ice to reach binding equilibrium. The following day, 100 mcl of hydroxylapatite slurry (50%) was added to each tube and mixed at 10-minute intervals for 30 minutes. The hydroxylapatite was collected by centrifugation and then washed 3 times with Tris-EDTA buffer (50 mM Tris, 1.5 mM EDTA, pH 7.4) containing 0.5% Titron X-100. After the final wash, the pellets were transferred to scintillation vials containing 4 ml of Biosafe II scintillation cocktail, mixed and placed in a scintillation counter. Total binding was determined from the tubes containing only radiolabeled ligand.

HL-60 Differentiation and Test Material. The drugs were dissolved in ethanol, and the concentration was determined using UV spectrophotometry. Serial dilutions were prepared so that a range of drug concentrations could be tested without changing the final concentration of ethanol (≦0.2%) present in the cell cultures. Human promyelocytic leukemia (“HL-60”) cells were grown in RPMI-1640 medium containing 10% fetal bovine serum. The cells were incubated at 37° C. in the presence of 5% CO₂. HL-60 cells were plated at 1.2×10⁵ cells/ml. Eighteen hours after plating, cells in duplicate were treated with the drug. Four days later, the cells were harvested, and a nitro blue tetrazolium reduction assay was performed (Collins et al, (1979); J. Exp. Med. 149:969-974). The percentage of differentiated cells was determined by counting a total of 200 cells and recording the number that contained intracellular black-blue formazan deposits. Verification of differentiation to monocytic cells was determined by measuring phagocytic activity.

In vitro Transcription Assay. Transcription activity was measured in ROS 17/2.8 bone cells that were stably transfected with a 24-hydroxylase (“24Ohase”) gene promoter upstream of a luciferase reporter gene (Arbour et al, (1998)). Cells were given a range of doses. Sixteen hours after dosing, the cells were harvested and luciferase activities were measured using a luminometer. (RLU=relative luciferase units).

Intestinal Calcium Transport and Bone. Calcium Mobilization. Male, weanling Sprague-Dawley rats were placed on a Diet 11 (0.47% Ca) diet+AEK for one week followed by Diet 11 (0.02% Ca)+AEK for 3 weeks. The rats were then switched to a diet containing 0.47% Ca for one week followed by two weeks on a diet containing 0.02% Ca. Administration of drug began during the last week on the 0.02% calcium diet. Four consecutive ip doses were given approximately 24 hours apart. 24 hours after the last dose, blood was collected from the severed neck, and the concentration of serum calcium was determined as a measure of bone calcium mobilization. The first 10 cm of the intestine was also collected for intestinal calcium transport analysis using the everted gut sac method. The everted sac method was carried out as described in Sicinski et al, J. Med. Chem. 41, 4662-4674 (1998).

The negative control material (“vehicle”) was prepared by volumetrically measuring ethanol (<5%) and propylene glycol, mixing and than placing in storage at 2-8° C.

Positive Control Material. 1,25(OH)₂D₃ was prepared by determining the concentration of an ethanol stock solution using UV spectrophotometry (extinction coefficient=18,200; λₘₐₓ=265 nm). The required amount of 1,25(OH)₂D₃ was volumetrically measured into propylene glycol so that there was less than 5% ethanol in the final solution. The solution was mixed and then stored at 2-8° C.

The instant vitamin D analogs were prepared by first determining the concentration of an ethanol stock solution using UV spectrophotometry (extinction coefficient=42,000, λₘₐₓ=252 nm). The analog solutions were than volumetrically added to propylene glycol so that there was less than 5% ethanol in the final solution. The solution was mixed and stored at 2-8° C.

Dose Administration Method. All experimental doses were administered by intraperitoneal injection in 100 microliters for 4-7 consecutive days spaced approximately 24 hours apart. 1,25(OH)₂D₃ was administered 4 consecutive days.

Serum Calcium Analysis. 24 hours after the final dose, approximately 1 ml of blood was allowed to coagulate at room temperature, and then centrifuged at 3000×g for 15 minutes. The serum was transferred to a polypropylene tube and stored frozen at −20° C. The level of calcium was determined by diluting the serum into 0.1% lanthum chloride. Absorbance was measured on an atomic absorption spectrophotometer, Perkin Elmer Model 3110 (Shelton, Conn.).

### Example I

Preparation of (20R)-1α-hydroxy-2-[3′-hydroxypropylidene]-19,24,25,26,27-pentanorvitamin D₃ (8a). Referring to SCHEME 1, the starting bicyclic aldehyde 1a was obtained according to the procedure set forth herein. (See Fall et al., Tetrahedron Lett. 43, 1433 (2002)).

(a) Wittig reaction of the aldehyde 1a. Benzoic acid (1R,3aR,4S,7aR)-7a-methyl-1-((R)-1-methyl-prop-2-enyl)-octahydro-inden-4-yl ester (2a). To the methyltriphenylphoshonium bromide (31 mg, 87 μmol) in anhydrous THF (0.5 mL) at 0° C. was added drop-wise n-BuLi (2.65 M in hexanes, 64 μL, 0.170 mmol) under argon with stirring. After 5 minutes, another portion of Ph₃P⁺CH₃ Br⁻ was added (31 mg, 87 μmol), and the solution was stirred at 0° C. for 10 minutes, and then at room temperature for 20 minutes. The orange-red mixture was cooled to negative 78° C. and siphoned to a solution of aldehyde 1a (33 mg, 0.109 mmol) in anhydrous THF (0.1 mL). The reaction mixture was stirred at −78° C. and stopped by addition of brine cont. 1% HCl three hours after adding of the first portion of the Wittig reagent. Ethyl acetate (3 mL), benzene (2 mL), ether (1 mL), saturated NaHCO₃ (1 mL), and, water (1 ml) were added, and the mixture was vigorously stirred at room temperature for 18 hours. Then, an organic phase was separated, washed with brine, dried (MgSO₄), and, evaporated. The oily residue was filtered through a silica Sep-Pak (2 g). Elution with hexane/ethyl acetate (99:1) resulted in pure olefinic product 2a (19 mg, 68%). 2a: [α]²⁴_{D} +71.0° (c 0.90 CHCl₃); ¹H NMR (400 MHz, CDCl₃) δ1.058 (3H, d, J=6.6 Hz, 21-H₃), 1.079 (3H, s, 18-H₃), 4.83 (1H, dd, J=10.1, 1.7 Hz, (23E)-H), 4.91 (1H, dd, J=17.2, 1.7 Hz, (23Z)-H), 5.41 (1H, narr m, 8α-H), 5.67 (1H, ddd, J=17.2, 10.1, 8.6 Hz, 22-H), 7.44 (2H, t, J=7.4 Hz, Ar—H), 7.55 (1H, t, J=7.4 Hz, Ar—H), 8.05 (2H, d, J=7.4 Hz, Ar—H); HRMS (ESI) exact mass calcd for C₁₇H₂₁O₂ (M⁺-C₆H₅CO) 257.1542, measured 257.1530.

(b) Hydrogenation of 22-olefin 2a. Benzoic acid (1R,3aR,4S,7aR)-1-((R)-sec-butyl)-7a-methyl-octahydro-inden-4-yl ester (3a). To a solution of olefin 2a (45 mg, 0.146 mmol) in ethyl acetate (5.5 mL) was added Pd/C (10%, 27 mg), and the resultant suspension was stirred under constant flow of hydrogen at room temperature for 19 hours. Then, the suspension was filtered. The filtrate was evaporated and applied to silica Sep-Pak cartridge (2 g). Elution with hexane/ethyl acetate (96:4) gave pure, oily ester 3a (40 mg, 87%). 3a: [α]²⁴_{D} +53.0° (c 0.58 CHCl₃); ¹H NMR (400 MHz, CDCl₃) δ 0.836 (3H, t, J=7.4 Hz, 23-H₃), 0.931 (3H, d, J=6.6 Hz, 21-H₃), 1.047 (3H, s, 18-H₃), 5.41 (1H, narr m, 8α-H), 7.45 (2H, t, J=7.4 Hz, Ar—H), 7.55 (1H, t, J=7.4 Hz, Ar—H), 8.06 (2H, d, J=7.4 Hz, Ar—H).

(c) Hydrolysis of the benzoate 3a. (1R,3aR,4S,7aR)-1-((R)-sec-Butyl)-7a-methyl-octahydro-inden-4-ol (4a). Solution of the ester 3a (40 mg, 0.129 mmol) in 10% methanolic KOH (2 mL) was heated at 50° C. for 24 hours, poured into water, and, extracted using ethyl acetate. The organic phase was washed with NaHCO₃ and water, and then dried (MgSO₄) and evaporated. The oily residue was purified using silica Sep-Pak (2 g). Elution with hexane/ethyl acetate (96:4) resulted in pure product 4a (22 mg, 81%). 4a: [α]²⁴_{D} +38° (c 1.0 CHCl₃); ¹H NMR (400 MHz, CDCl₃) δ 0.824 (3H, t, J=7.4 Hz, 23-H₃), 0.821 (3H, d, J=6.5 Hz, 21-H₃), 0.931 (3H, s, 18-H₃), 4.08 (1H, narr m, 8α-H); HRMS (ESI) exact mass calcd for C₁₄H₂₆O (M⁻) 210.1984, measured 210.1990.

(d) Oxidation of alcohol 4a. (1R,3aR,4S,7aR)-1-((R)-sec-Butyl)-7a-methyl-octahydro-inden-4-one (5a). A solution of NMO (23 mg) and molecular sieves 4 Å (138 mg) in methylene chloride (0.9 mL) was stirred at room temperature for 15 minutes. The solution of 4a (21 mg, 0.10 mmol) in methylene chloride (0.15 mL) was added followed by TPAP (2.5 mg). The resultant dark mixture was stirred for 30 minutes and applied to a silica Sep-Pak (2 g). Elution using hexane/ethyl acetate (95:5) produced pure ketone 5a (18.5 mg, 88%). 5a: [α]²⁴_{D}-11° (c 0.78 CHCl₃), ¹H NMR (400 MHz, CDCl₃) δ 0.640 (3H, s, 18-H₃), 0.843 (3H, t, J=7.4 Hz, 23-H₃), 0.945 (3H, d, J=5.9 Hz, 21-H₃), 2.12 (1H, br d, J=12.8 Hz, 9β-H), 2.45 (1H, dd, J=11.6, 7.6 Hz, 14α-H); HRMS (ESI) exact mass calcd for C₁₄H₂₄O (M⁻) 208.1827, measured 208.1830.

(e) Wittig-Horner coupling of the ketone 5a with the phosphine oxide 6. 1α-[(tert-Butyldimethylsilyl)oxy]-2-[3′-[((tert-butyldimethylsilyl)oxy)propylidene]-19,24,25,26,27-pentanorvitamin D₃ tert-Butyldimethylsilyl Ether (E-isomer, 7a). To a solution of phosphine oxide 6 (11.5 mg, 15.6 μmol) in anhydrous THF (0.30 mL) at −78° C. phenyllithium (1.8 M in butyl ether, 9 μL, 16 μmol) was slowly added under argon with stirring. The solution turned deep orange. The mixture was stirred at −78° C. for 20 minutes. A pre-cooled (−78° C.) solution of ketone 5a (19 mg, 91 μmol) in anhydrous THF (0.10 mL) was slowly added. The mixture was stirred under argon at −78° C. for 2 hours and at 6° C. for 16 hours. Ethyl acetate and water were added, and the organic phase was washed with brine, dried with MgSO₄, and evaporated. The residue was dissolved in hexane, applied on a silica column, and washed with hexane/ethyl acetate (98.5:1.5) to produce silylated vitamin 7a (1.44 mg, 13%). The column was then washed with hexane/ethyl acetate (95.5) to recover a portion of unchanged C,D-ring ketone 5a (7 mg), and hexane/ethyl acetate (6:4) was used to recover diphenylphosphine oxide 6 (4.2 mg). 7a: ¹H NMR (400 MHz, CDCl₃) δ-0.023, 0.051, 0.050, 0.059 and 0.069 (3H, 3H, 3H, 3H and 6H, each s, 6×SiCH₃), 0.549 (3H, s, 18-H₃), 0.819, 0.896 and 0.923 (each 9H, each s, 3×Si-t-Bu), 2.33 (2H, m, ═CH—CH₂), 2.79 (1H, dd, J˜12.5, 3 Hz, 9β-H), 3.05 (1H, dd, J=12.5, 4.4 Hz, 10β-H), 3.62 (2H, m, CH₂—CH₂—O), 4.34 (1H, m, w/2=21 Hz, 1β-H), 4.81 (1H, narr m, 3α-H), 5.47 (1H, t, J=7.5 Hz, ═CH—CH₂), 5.87 and 6.11 (1H and 1H, each d, J=10.9 Hz, 7- and 6-H); HRMS (ESI) exact mass calcd for C₄₃H₈₂O₃Si₃Na (M⁻+Na) 753.5470, fd 753.5465.

(f) Hydrolysis of the silyl protecting groups in the 19-norvitamin D derivative 7a. 1α-Hydroxy-2-[3′-hydroxypropylidene]-19,24,25,26,27-pentanorvitamin D₃ (E-isomer, 8a). To a solution of the protected vitamin 7a (1.4 mg, 1.91 μmol) in anhydrous THF (1.3 mL), tetrabutylammonium fluoride (1.0 M in THF, 86 μL, 86 μmol) and triethylamine (16 μL) were added. The mixture was stirred under argon at room temperature for 18 hours, poured into brine, and, extracted using ethyl acetate and diethyl ether. Organic extracts were washed with brine, dried using MgSO₄, and evaporated. The residue was purified using HPLC (9.4 mm×25 cm Zorbax-Sil column, 4 mL/min) using hexane/2-propanol (8:2) solvent system. Pure 19-norvitamin 8a (0.56 mg, 72%) was collected at Rᵥ 25.5 mL. In a reversed-phase HPLC (9.4 mm×25 cm Eclipse XDB-C18 column, 4 mL/min) using methanol/water (9:1) solvent system, vitamin 8a was collected at Rᵥ 42 mL. 8a (“RBH”): UV (in EtOH)) λₘₐₓ 243.0, 251.5, 261.5 nm; ¹H NMR (400 MHz, CDCl₃) δ0.549 (3H, s, 18-H₃), 0.917 (3H, br d, J=5.5 Hz, 21-H₃), 0.837 (3H, t, J=7.4 Hz, 23-H₃), 2.47 (2H, narr m, 4α- and 4β-H), 2.36 and 2.54 (1H and 1H, each m, ═CH—CH₂), 2.82 (1H, dm, J˜13.5 Hz, 9β-1H), 3.16 (1H, dd, J=13.2, 5.0 Hz, 10β-H), 3.66 and 3.76 (1H and 1H, each m, CH₂—CH₂—O), 4.44 (1H, m, w/2=20 Hz, 1β-H), 4.85 (1H, narr m, 3α-H), 5.67 (1H, t, J=7.5 Hz, ═CH—CH₂), 5.88 and 6.31 (1H and 1H, each d, J=11.6 Hz, 7- and 6-H); HRMS (ESI) C₂₅H₄₀O₃Na (M⁺+Na) 411.3079, measured 411.3086.

### Example II

Preparation of (20S)-1α-hydroxy-2-[3′-hydroxypropylidene]-19,24,25,26,27-pentanorvitamin D₃ (8b). As set forth in SCHEME II, starting bicyclic aldehyde 2b was obtained according to the procedure set forth in Granja et al., J. Org. Chem. 58, 124 (1993).

(a) Wittig reaction of the aldehyde 2b. Benzoic acid (1R,3aR,4S,7aR)-7a-methyl-1-((S)-1-methyl-prop-2-enyl)-octahydro-inden-4-yl ester (2b). To methyltriphenylphoshonium bromide (63 mg, 0.179 mmol) in anhydrous THF (0.5 mL) at 0° C., n-BuLi (2.65 M in hexanes, 128 μL, 0.340 mmol) was added drop-wise under argon with stirring. After 5 minutes, another portion of Ph₃P⁺CH₃ Br⁻ was added (63 mg, 0.179 mmol), and the solution was stirred at 0° C. for 10 minutes and at room temperature for 20 minutes. The orange-red mixture was cooled to negative 78° C. and siphoned to produce a solution of aldehyde 1b (56 mg, 0.185 mmol) in anhydrous THF (0.2 mL). The reaction mixture was stirred at −78° C. and stopped by adding brine cont. 1% HCl three hours after addition of the first portion of the Wittig reagent. Ethyl acetate (3 mL), benzene (2 mL), ether (1 mL), saturated NaHCO₃ (1 mL), and water (1 ml) were added, and the mixture was vigorously stirred at room temperature for 18 hours. An organic phase was separated, washed with brine, dried with MgSO₄, and evaporated. The oily residue was filtered through a silica Sep-Pak (2 g). Elution using hexane/ethyl acetate (98:2) resulted in pure olefinic product 2b (46 mg, 73%). 2b: [α]²⁴_{D} +12° (c 0.39 CHCl₃); ¹H NMR (400 MHz, CDCl₃) δ 0.940 (3H, d, J=6.6 Hz, 21-H₃), 1.046 (3H, s, 18-H₃), 4.87 (1H, dd, J=10.1, 1.6 Hz, (23E)-H), 4.97 (1H, dd, J=17.1, 1.6 Hz, (23Z)-H), 5.41 (1H, narr m, 8α-H), 5.70 (1H, dt, J=17.1, 9.7 Hz, 22-H), 7.44 (2H, t, J=7.3 Hz, Ar—H), 7.55 (1H, t, J=7.3 Hz, Ar—H), 8.05 (2H, d, J=7.3 Hz, Ar—H, HRMS (ESI) exact mass calcd for C₁₇H₂₁O₂C₁₇H₂₁O₂ (M⁻-C₆H₅CO) 257.1542, measured 257.1533.

(b) Hydrogenation of 22-olefin 2b. Benzoic acid (1R,3aR,4S,7aR)-1-((S)-sec-butyl)-7a-methyl-octahydro-inden-4-yl ester (3b). To a solution of olefin 2b (47 mg, 0.153 mmol) in ethyl acetate (5.7 mL), Pd/C (10%, 28 mg) was added, and the resultant suspension was stirred under constant flow of hydrogen at room temperature for 20 hours. The suspension was filtered. The filtrate was evaporated and applied to silica Sep-Pak cartridge (2 g). Elution using hexane/ethyl acetate (97:3) produced pure, oily ester 3b (40.5 mg, 86%). 3b: [α]²⁴_{D} +49.0° (c 0.37 CHCl₃); ¹H NMR (400 MHz, CDCl₃) δ) 0.829 (3H, d, J=6.6 Hz, 21-H₃), 0.850 (3H, t, J=7.4 Hz, 23-H₃), 1.045 (3H, s, 18-H₃), 5.41 (1H, narr m, 8α-H), 7.44 (2H, t, J=7.6 Hz, Ar—H), 7.55 (1H, tt, J=7.4, ˜1.4 Hz, Ar—H), 8.06 (2H, m, Ar—H).

(c) Hydrolysis of the benzoate 3b. (1R,3aR,4S,7aR)-1-((S)-sec-Butyl)-7a-methyl-octahydro-inden-4-ol (4b). Solution of the ester 3b (40.5 mg, 0.131 mmol) in 10% methanolic KOH (2 mL) was heated at 50° C. for 23 hours, poured into water, and, extracted using ethyl acetate. The organic phase was washed using NaHCO₃ and water, dried using MgSO₄, and evaporated. The oily residue was purified using silica Sep-Pak (2 g). Elution using hexane/ethyl acetate (97:3) produced pure product 4b (22 mg, 80%). 4b: [α]²⁴_{D} +25° (c 0.29 CHCl₃), ¹H NMR (400 MHz, CDCl₃) δ 0.822 (3H, t, J=7.6 Hz, 23-H₃), 0.813 (3H, d, J=7.3 Hz, 21-H₃), 0.929 (3H, s, 18-H₃), 4.07 (1H, narr m, 8α-H); HRMS (ESI) exact mass calcd for C₁₄H₂₆O (M⁺) 210.1984, measured 210.1984.

(d) Oxidation of alcohol 4b. (1R,3aR,4S,7aR)-1-((S)-sec-Butyl)-7a-methyl-octahydro-inden-4-one (5b). A solution of NMO (28 mg) and molecular sieves 4 Å (145 mg) in methylene chloride (0.9 mL) was stirred at room temperature for 15 minutes. The solution of 4b (22 mg, 0.104 mmol) in methylene chloride (0.15 mL) was added followed by TPAP (3.0 mg). The resultant dark mixture was stirred for 30 minutes, and applied to a silica Sep-Pak (2 g). Elution with hexane/ethyl acetate (96:4) produced pure ketone 5b (18.0 mg, 82%). 5b: [α]²⁴_{D}-27.5° (c 0.8 CHCl₃); ¹H NMR (400 MHz, CDCl₃) δ 0.636 (3H, s, 18-H₃), 0.857 (3H, t, J=7.4 Hz, 23-H₃), 0.848 (3H, d, J˜7 Hz, 21-H₃), 2.09 (1H, br d, J=12.0 Hz, 9β-H), 2.45 (1H, dd, J=11.5, 7.6 Hz, 14α-H), HRMS (ESI) exact mass calcd for C₁₄H₂₄O (M⁻) 208.1827, measured 208.1836.

(e) Wittig-Horner coupling of the ketone 5b with the phosphine oxide 6. (20S)-1α-[(tert-Butyl dimethylsilyl)oxy]-2-[3′-[((tert-butyldimethylsilyl)oxy)propylidene]-19,24,25,26,27-pentanorvitamin D₃ tert-Butyldimethylsilyl Ether (E-isomer, 7b). To a solution of phosphine oxide 6 (11.5 mg, 15.6 μmol) in anhydrous THF (0.30 mL) at −78° C., phenyllithium (1.8 M in butyl ether, 9 μL, 16 μmol) was slowly added under argon with stirring. The solution turned deep orange. The mixture was stirred at −78° C. for 20 minutes. A pre-cooled (−78° C.) solution of the ketone 5b (19 mg, 91 μmol) in anhydrous THF (0.10 mL) was slowly added. The mixture was stirred under argon at −78° C. for 2 hours and at 6° C. for 16 hours. Ethyl acetate and water were added. The organic phase was washed with brine, dried using MgSO₄, and evaporated. The residue was dissolved in hexane, applied on a silica column, and, washed using hexane/ethyl acetate (98.5:1.5) producing silylated vitamin 7b (2.2 mg, 19%). The column was washed using hexane/ethyl acetate (96:4) to recover a portion of unchanged C,D-ring ketone 5b (9 mg), and hexane/ethyl acetate (6:4) was used to recover diphenylphosphine oxide 6 (4 mg). 7b: UV (in hexane) λₘₐₓ 243.5, 252.5, 262.0 nm; ¹H NMR (400 MHz, CDCl₃) δ-0.023, 0.055, 0.059, and 0.069 (3H, 3H, 6H, and 6H, each s, 6×SiCH₃), 0.552 (3H, s, 18-H₃), 0.819, 0.896, and 0.923 (each 9H, each s, 3×Si-t-Bu), 2.36 and 2.54 (1H and 1H, each m, ═CH—CH₂), 2.79 (1H, br d, J˜12.7 Hz, 9β-H), 3.05 (1H, dd, J˜12, 5.0 Hz, 10β-H), 3.63 (2H, m, CH₂—CH₂—O), 4.34 (1H, m, w/2=21 Hz, 1β-H), 4.81 (1H, narr m, 3α-H), 5.47 (1H, t, J=7.5 Hz, HC═C—CH₂), 5.85 and 6.13 (1H and 1H, each d, J=11.6 Hz, 7- and 6-H); HRMS (ESI) exact mass calculated for C₄₃H₈₂O₃Si₃Na (M⁺+Na) 753.5470, measured 753.5462.

(f) Hydrolysis of the silyl protecting groups in the 19-norvitamin D derivative 7b. (20S)-1α-Hydroxy-2-[3′-hydroxypropylidene]-19,24,25,26,27-pentanorvitamin D₃ (E-isomer, 8b). To a solution of the protected vitamin 7b (2.2 mg, 3.01 μmol) in anhydrous THF (2 mL), tetrabutylammonium fluoride (1.0 M in THF, 135 μL, 135 μmol) and triethylamine (25 μL) were added. The mixture was stirred under argon at room temperature for 18 hours, poured into brine, and, extracted using ethyl acetate and diethyl ether. Organic extracts were washed using brine, dried using MgSO₄, and evaporated. The residue was purified using HPLC (9.4 mm×25 cm Zorbax-Sil column, 4 mL/min) using hexane/2-propanol (8:2) solvent system. Pure 19-norvitamin 8b (0.66 mg, 53%) was collected at Rᵥ 25.5 mL. In reversed-phase HPLC (9.4 mm×25 cm Eclipse XDB-C18 column, 4 mL/min) using methanol/water (95:5) solvent system, vitamin 8b was collected at Rᵥ 42 mL. 8b (“SBH”): UV (in EtOH) λₘₐₓ 243.0, 251.5, 261.5 nm, ¹H NMR (400 MHz, CDCl₃) δ 0.545 (3H, s, 18-H₃), 0.835 (3H, d, J=5.8 Hz, 21-H₃), 0.836 (3H, t, J=7.3 Hz, 23-H₃), 2.47 (2H, narr m, 4α- and 4β-H), 2.36 and 2.55 (1H and 1H, each m, ═CH—CH₂), 2.82 (1H, br d, J=12.9 Hz, 9β-H), 3.16 (1H, dd, J=13.2, 5.0 Hz, 10β-H), 3.66 and 3.76 (1H and 1H, each m, CH₂—CH₂—O), 4.45 (1H, in, w/2=20 Hz, 1β-H), 4.85 (1H, narr m, 3α-H) 5.67 (1H, t, J=7.5 Hz, ═CH—CH₂), 5.88 and 6.31 (1H and 1H, each d, J=11.6 Hz, 7- and 6-H), HRMS (ESI) exact mass calcd for C₂₅H₄₀O₃Na (M⁺+Na) 411.3079, measured 411.3089.

### Example III

Preparation of (20R)-1α-hydroxy-2-[3′-hydroxypropylidene]-19,23,24-trinorvitamin D₃ (15a) and (20S)-1α-hydroxy-2-[3′-hydroxypropylidene]-19,23,24-trinorvitamin D₃ (15b).

(a) Conversion of aldehyde 1a into 22-nitrile 9. Benzoic acid-(1R,3aR,4S,7aR)-1-((R-cyano-methyl-methyl)-7a-methyl-octahydro-inden-4-yl ester (9). To a solution of a benzoyloxy aldehyde 1a (284 mg, 0.90 mmol) in anhydrous pyridine (5 mL), NH₂OH×HCl (210 mg) was added. The mixture was stirred at room temperature for 20 hours. The mixture was poured into water and extracted using ethyl acetate. The combined organic phases were separated, washed using saturated NaHCO₃ solution, water, and saturated CuSO₄ solution, dried using MgSO₄, and evaporated. The oily residue was purified using column chromatography on silica gel. Elution using hexane/ethyl acetate (9:1) produced pure (less polar)/E-oxime (167 mg) and (more) polar Z-oxime (105 mg, total yield 89%). E-oxime: ¹H NMR (400 MHz, CDCl₃) δ 1.09 (3H, d, J=6.7 Hz, 18-H₃), 1.14 (3H, s, 21-H₃), 2.40 (1H, m, 20-H), 5.42 (1H, narr m, 8α-H), 7.27 (1H, d, J=8.0 Hz, 22-H), 7.45 (2H, t, J˜7 Hz, Ar—H), 7.56 (1H, t, J=7.4 Hz, Ar—H), 8.04 (2H, d, J=7.4 Hz, Ar—H). Z-oxime: ¹H NMR (400 MHz, CDCl₃) δ 1.09 (3H, d, J=6.7 Hz, 18-H₃), 1.13 (3H, s, 21-H₃), 3.28 (1H, m, 20-H), 5.42 (1H, narr m, 8α-H), 6.25 (1H, d, J=8.1 Hz, 22-H), 7.45 (2H, t, J˜7 Hz, Ar—H), 7.56 (1H, t, J=7.3 Hz, Ar—H), 8.04 (2H, d, J=7.3 Hz, Ar—H).

The solution of oximes (both isomers, 248 mg, 0.75 mmol) in acetic anhydride (8 mL) was refluxed for 1.5 hours. The reaction mixture was cooled, poured carefully on ice, and, extracted using toluene. Extracts were combined, washed with water, NaHCO₃ and brine, dried using MgSO₄, and evaporated. The residue was applied to silica Sep-Pak (5 g). Elution with hexane/ethyl acetate (95:5) produced pure semi-crystalline nitrile 9 (212 mg, 91%). 9: [α]²⁴_{D} +81.5° (c 0.9 CHCl₃); ¹H NMR (400 MHz, CDCl₃) δ 1.124 (3H, s, 18-H₃), 1.373 (3H, d, J=7.1 Hz, 21-H₃), 1.90 (1H, br d, J=12.8 Hz, 9β-H), 2.68 (1H, pentet, J=7.0 Hz, 20-H), 5.43 (1H, narr m, 8α-H), 7.45 (2H, t, J=7.6 Hz, Ar—H), 7.57 (1H, t, J=7.5 Hz, Ar—H), 8.03 (2H, d, J=7.4 Hz, Ar—H); HRMS (ESI) exact mass calcd for C₁₃H₂₀ON (M⁺-C₆H₅CO) 206.1545, measured 206.1539.

(b) Alkylation of the nitrile 9 with iso-butyl bromide. Benzoic acid-(1 S,3aR,4S,7aR)-1-((S)-1-cyano-1,3-dimethyl-butyl)-7a-methyl-octahydro-inden-4-yl ester (10). n-BuLi (2.65 M in hexanes, 103 μL, 0.272 mmol) was added at 0° C. to the flask containing diisopropylamine (42 μL, 0.272 mmol) and THF (0.4 mL). The solution was stirred at 0° C. for 20 minutes, cooled to negative 78° C., and, siphoned to produce a solution of 9 (77 mg, 0.248 mmol) in THF (0.3 mL). The resultant yellow mixture was stirred for 30 minutes. HMPA (100 μL) was added. Stirring continued for another 15 minutes. (CH₃)₂CHCH₂Br (68 μL, 0.62 mmol) was added. The solution was allowed to warm up to 40° C. over a duration of 1 hour. Saturated NH₄Cl was added. The mixture was extracted using ethyl acetate. The combined organic phases were washed with water, dried using MgSO₄, and evaporated. The residue was applied to silica SepPak (2 g). Elution using hexane/ethyl acetate (98:2) resulted in pure semi-crystalline 10 (60 mg, 66%, 74% based on recovered substrate); further elution with hexane/ethyl acetate (97:3) gave un-reacted 9 (8.5 mg). 10: [α]²⁴_{D} +66.5° (c 1.15 CHCl₃). ¹H NMR (400 MHz, CDCl₃) δ 1.055 and 0.971 (3H and 3H, each d, J=6.6 Hz, 24- and 25-H₃), 1,369 (3H, s, 18-H₃). 1.456 (3H, s, 21-H₃), 2.15 (1H, br d, J=12.7 Hz, 9β-H), 5.40 (1H, narr m, 8α-H), 7.45 (2H, t, J˜7 Hz, Ar—H), 7.57 (1H, t, J=7.4 Hz, Ar—H), 8.04 (2H, d, J=7.4 Hz, Ar—H); HRMS (ESI) exact mass calculated for C₂₄H₃₃O₂N (M⁺) 367.2511, measured 367.2518.

(c) Hydrolysis of the 8β-benzoyloxy group in the nitrile 10. (S)-2-((1S,3aR,4S,7aR)-4-hydroxy-7a-methyl-octahydro-inden-1-yl)-2,4-dimethyl-pentanenitrile (11). Benzoyloxy nitrile 10 (90 mg, 0.246 mmol) was treated using 10% methanolic KOH (4 mL) at 50° C. for 18 hours. After concentration under vacuum, the reaction mixture was poured into water and extracted using benzene and ether. The organic extracts were combined, washed with brine, dried using MgSO₄, and evaporated. The residue was re-dissolved in hexane/ethyl acetate (95:5). The solution was passed through a silica gel Sep-Pak cartridge. Evaporation of solvents produced hydroxy nitrile 11 (66 mg, 92%). 11: [α]²⁴_{D} +28° (c 0.29 CHCl₃); ¹H NMR (400 MHz, CDCl₃) δ 1.043 and 0.959 (3H and 3H, 2 x d, J=6.6 Hz, 24-H₃ and 25-H₃), 1.236 (3H, s, 18-H₃), 1.410 (3H, s, 21-H₃), 2.08 (1H, d m, J=12.4 Hz, 9β-H), 4.09 (1H, narr m, 8α-H), HRMS (ESI) exact mass calcd for C₁₇H₂₉ON (M⁺) 263.2249, measured 263.2254.

(d) Reductive decyanation of hydroxy nitrile 11. (1R,3aR,4S,7aR)-1-((R)-1,3-Dimethyl-butyl)- and (1R,3aR,4S,7aR)-1-((S)-1,3-Dimethyl-butyl)-7a-methyl-octahydro-inden-4-ol (12a,b). A solution of nitrile 11 (49 mg, 0.186 mmol) in t-BuOH (50 μL) and ether (0.20 mL) was added drop-wise at 0° C., under argon, to a blue solution of potassium (55 mg, 1.4 mmol) in HMPA (0.17 mL) and ether (0.42 mL). A cooling bath was removed, and stirring continued for 4 hours at room temperature under argon. The reaction mixture was diluted using benzene. Un-reacted potassium was removed and, a few drops of 2-propanol were added. The organic phase was washed using water, dried using MgSO₄, and evaporated. The residue was applied to a silica Sep-Pak (2 g). Elution using hexane/ethyl acetate (95:5) produced a 1:1 mixture of epimeric alcohols 12a and 12b (37 mg, 84%). 12a and 12b: ¹H NMR (400 MHz, CDCl₃, selected signals) δ 0.932 (s, 18-H₃ in 12b), 0.944 (s, 18-H₃ in 12a), 2.01 (br d. J=12.7 Hz, 9β-H from both isomers), 4.07 (narr m, 8α-H from both isomers); HRMS (ESI) exact mass calculated for C₁₆H₃₀O (M⁺) 238.2297, measured 238.2294.

(e) Oxidation of alcohols 12a and 12b. (1R,3aR,7aR)-1-((R)-1,3-dimethyl-butyl)- and (1R,3aR,7aR)-1-((S)-1,3-dimethyl-butyl)-7a-methyl-octahydro-inden-4-one (13 a and 13b). The solution of NMO (23 mg) and molecular sieves 4 Å (123 mg) in methylene chloride (0.9 mL) was stirred at room temperature for 15 minutes. The solution of 12a and 12b (20.5 mg, 86 μmol) in methylene chloride (0.15 mL) was added followed by the TPAP (2.5 mg). The resultant dark mixture was stirred for 30 minutes, diluted with methylene chloride, and filtered through silica SepPak (2 g). Elution using methylene chloride produced a 1:1 mixture of epimeric ketones 13a and 13b (21 mg, 91%). Separation of isomers was achieved using HPLC (9.4 mm×25 cm Zorbax-Sil column, 4 mL/min) using hexane/ethyl acetate (95:5) solvent system. The 20S ketone 13b was collected at Rᵥ 39 mL and the R-isomer 13a at Rᵥ 40 mL. 13a: [α]²⁴_{D} +11° (c 0.28 CHCl₃); ¹H NMR (400 MHz, CDCl₃) δ 0.653 (3H, s, 18-H₃), 0.816 and 0.881 (3H and 3H, each d, J=6.6 Hz, 24- and 25-H₃), 0.922 (3H, d, J=5.9 Hz, 21-H₃), 2.14 (1H, br d, J=12.4 Hz, 9β-H), 2.44 (1H, dd, J=11.6, 7.6 Hz, 14α-H); HRMS (ESI) exact mass calcd for C₁₆H₂₈O (M⁺) 236.2140, measured 236.2135. 13b: [α]²⁴_{D}−48° (c 0.28 CHCl₃), ¹H NMR (400 MHz, CDCl₃) δ 0.641 (3H, s, 18-H₃), 0.827 and 0.831 (3H and 3H, each d, J=6.6 Hz, 24- and 25-H₃), 0.894 (3H, d, J=5.9 Hz, 21-H₃), 2.12 (1H, br d, J=12.7 Hz, 9β-H), 2.44 (1H, dd, J=11.5, 7.6 Hz, 14α-H); HRMS (ESI) exact mass calculated for C₁₆H₂₈O (M⁻) 236.2140, measured 236.2135.

(f) Wittig-Horner coupling of the ketones 13a,b with the phosphine oxide 6. (a) 1α-[(tert-Butyldimethylsilyl)oxy]-2-[3′-[((tert-butyldimethylsilyl)oxy)propylidene]-19,23,24-trinorvitamin D₃ tert-Butyldimethylsilyl Ether (E-isomer, 14a). To a solution of phosphine oxide 6 (28 mg, 38 μmol) in anhydrous THF (0.40 mL) at −78° C., phenyllithium (1.8 M in butyl ether, 22 μL, 39 μmol) was slowly added under argon with stirring. The solution turned deep orange. The mixture was stirred at −78° C. for 20 minutes, and a pre-cooled (−78° C.) solution of the ketone 13a (7.5 mg, 32 μmol) in anhydrous THF (0.10 mL) was slowly added. The mixture was stirred under argon at −78° C. for 2 hours and at 6° C. for 16 hours. Ethyl acetate and water were added, and the organic phase was washed using brine, dried using MgSO₄, and evaporated. The residue was dissolved in hexane, applied on a silica Sep-Pak cartridge, and eluted using hexane/ethyl acetate (95.5:0.5) to produce 19-norvitamin derivative 14a (12 mg, 50%). The Sep-Pak was washed using hexane/ethyl acetate (98:2) to recover a portion of unchanged C,D-ring ketone 13a (1 mg), and with hexane/ethyl acetate (6:4) to recover diphenylphosphine oxide 6 (3 mg). 14a: ¹H NMR (400 MHz, CDCl₃) δ-0.023, 0.052, 0.056, 0.059 0.062 and 0.069 (each 3H, each s, 6×SiCH₃), 0.567 (3H, s, 18-H₃), 0.818, 0.897, and 0.923 (each 9H, each s, 3×Si-t-Bu) 2.37 (2H, each m, ═CH—CH₂), 2.79 (1H, br d, J=12.5 Hz, 9β-H), 3.05 (1H, dd, J=12.4, 4.4 Hz, 10β-H), 3.62 (2H, each in, CH₂—CH₂—O), 4.34 (1H, m, w/2=20 Hz, 1β-H), 4.80 (1H, narr m, 3α-H), 5.47 (1H, t, J˜7 Hz, ═CH—CH₂), 5.87 and 6.11 (1H and 1H, each d, J=11.1 Hz, 7- and 6-H).

(b) (20S)-1α-[(tert-Butyldimethylsilyl)oxy]-2-[3′-[((tert-butyldimethyl silyl)oxy) propylidene]-19,23,24-trinorvitamin D₃ tert-Butyldimethylsilyl Ether (E-isomer, 14b). To a solution of phosphine oxide 6 (27.5 mg, 37 μmol) in anhydrous THF (0.40 mL at −78° C., phenyllithium (1.8 M in butyl ether, 21 μL, 38 μmol) was slowly added under argon with stirring. The solution turned deep orange. The mixture was stirred at −78° C. for 20 minutes, and a pre-cooled (−78° C.) solution of the ketone 13b (7.0 mg, 30 μmol) in anhydrous THF (0.10 mL) was slowly added. The mixture was stirred under argon at −78° C. for 2 hours and at 6° C. for 16 hours. Ethyl acetate and water were added, and the organic phase was washed using brine, dried using MgSO₄, and evaporated. The residue was dissolved in hexane, applied to a silica Sep-Pak cartridge, and eluted using hexane/ethyl acetate (95.5:0.5) to produce 19-norvitamin derivative 14b (12 mg, 53%). The Sep-Pak was then washed using hexane/ethyl acetate (98:2) to recover a portion of unchanged C,D-ring ketone 13b (1 mg), and with hexane/ethyl acetate (6:4) to recover diphenylphosphine oxide 6 (2 mg). 14b: ¹H NMR (400 MHz, CDCl₃) δ-0.023, 0.056, 0.060, 0.071 and 0.088 (3H, 3H, 6H, 3H and 3H, each s, 6×SiCH₃), 0.551 (3H, s, 18-H₃), 0.819, 0.897, and 0.924 (each 9H, each s, 3×Si-t-Bu), 2.33 (2H, each m, ═CH—CH₂), 2.79 (1H, br d, J=12.4 Hz, 9β-H), 3.04 (1H, dd, J=12.4, 4.4 Hz, 10β-H), 3.62 (2H, each m, CH₂—CH₂—O), 4.34 (1H, m, w/2=20 Hz, 1β-H), 4.81 (1H, narr m, 3α-H), 5.47 (1H, t, J˜7 Hz, ═HC—CH₂), 5.87 and 6.12 (1H and 1H, each d, J=11.1 Hz, 7- and 6-H).

(g) Hydrolysis of the silyl protecting groups in the 19-norvitamin D derivatives 14a,b. (a) (20R)-1α-Hydroxy-2-[3′-hydroxypropylidene]-19,23,24-trinorvitamin D₃ (E-isomer, 15a). To a solution of the protected vitamin 14a (11.5 mg, 15 μmol) in anhydrous THF (9.5 mL), tetrabutylammonium fluoride (1.0 M in THF, 450 μL, 450 μmol) and triethylamine (84 μL) were added. The mixture was stirred under argon at room temperature for 18 hours, poured into brine, and extracted using ethyl acetate and diethyl ether. Organic extracts were washed using brine, dried using MgSO₄, and evaporated. The residue was purified using HPLC (9.4 mm×25 cm Zorbax-Sil column, 4 mL/min) using hexane/2-propanol (7:3) solvent system. Pure 19-norvitamin 15a (6.2 mg, 98%) was collected at Rᵥ 24 mL. In reversed-phase HPLC (9.4 mm×25 cm Eclipse XDB-C18 column, 4 mL/min) using, methanol/water (95:5) solvent system, vitamin 15a was collected at Rᵥ 31.5 mL. 15a (“HPBR”): UV (in EtOH), λₘₐₓ 243.0, 251.0, 261.0 nm, ¹H NMR (400 MHz, CDCl₃) δ 0.600 (3H, s, 18-H₃), 0.894 (3H, d, J=6.0 Hz, 21-H₃), 0.820 and 0 879 (1H and 1H, each d, J=6.4 Hz, 24- and 25-H₃), 2.44 (2H, narr m, 4α- and 4β-H), 2.31 and 2.52 (1H and 1H, each m, ═CH—CH₂), 2.81 (1H, br d, J=12.7 Hz, 9β-H), 3.15 (1H, dd, J=13.0, 4.8 Hz, 10β-H), 3.65 and 3.74 (1H and 1H, each m, CH₂—CH₂—O), 4.41 (1H, m, w/2=20 Hz, 1β-H), 4.82 (1H, narr m, 3α-H), 5.62 (1H, t, J=7.3 Hz, HC═C—CH₂), 5.88 and 6.30 (1H and 1H, each d, J=11.2 Hz, 7- and 6-H), HRMS (ESI) exact mass calculated for C₂₇H₄₄O₃Na (M⁺+Na) 439.3188, measured 439.3177.

(b) (20S)-1α-Hydroxy-2-[3′-hydroxypropylidene]-19,23,24-trinorvitamin D₃ (E-isomer, 15b). To a solution of the protected vitamin 14b (11.5 mg, 15 μmol) in anhydrous THF (9.5 mL), tetrabutylammonium fluoride (1.0 M in THF, 450 μL, 450 μmol) and triethylamine (84 μL) were added. The mixture was stirred under argon at room temperature for 18 hours, poured into brine, and extracted using ethyl acetate and diethyl ether. Organic extracts were washed using brine, dried using MgSO₄, and evaporated. The residue was purified using HPLC (9.4 mm×25 cm Zorbax-Sil column, 4 mL/min) using hexane/2-propanol (7:3) solvent system. Pure 19-norvitamin 15b (6.2 mg, 98%) was collected at Rᵥ 24 mL. In reversed-phase HPLC (9.4 mm×25 cm Eclipse XDB-C18 column, 4 mL/min) using methanol/water (95:5) solvent system, vitamin 15b was collected at Rᵥ 30 mL. 15b (“HPBS”): UV (in EtOH) λₘₐₓ 243.0, 251.0, 261.0 nm; ¹H NMR (400 MHz, CDCl₃) δ 0.546 (3H, s, 18-H₃), 0.879 (3H, d, J=6.5 Hz, 21-H₃), 0.815 and 0 824 (3H and 3H, each d, J=6.2 Hz and J=6.3 Hz, 24- and 25-H₃), 2.46 (2H, narr m, 4α- and 4β-H), 2.33 and 2.54 (1H and 1H, each m, ═CH—CH₂), 2.81 (1H, br d, J=12.7 Hz, 9β-H), 3.15 (1H, dd, J=13.0, 4.8 Hz, 10β-H), 3.67 and 3.73 (1H and 1H, each m, CH₂—CH₂—O), 4.42 (1H, m, w/2=20 Hz, 1β-H), 4.84 (1H, narr m, 3α-H), 5.65 (1H, t, J=7.3 Hz, ═CH—CH₂), 5.88 and 6.30 (1H and 1H, each d, J=11.2 Hz, 7- and 6-H), HRMS (ESI) exact mass calculated for C₂₇H₄₄O₃Na (M⁺+Na) 439.3188, measured 439.3180.

SCHEME I and SCHEME II are set forth below.

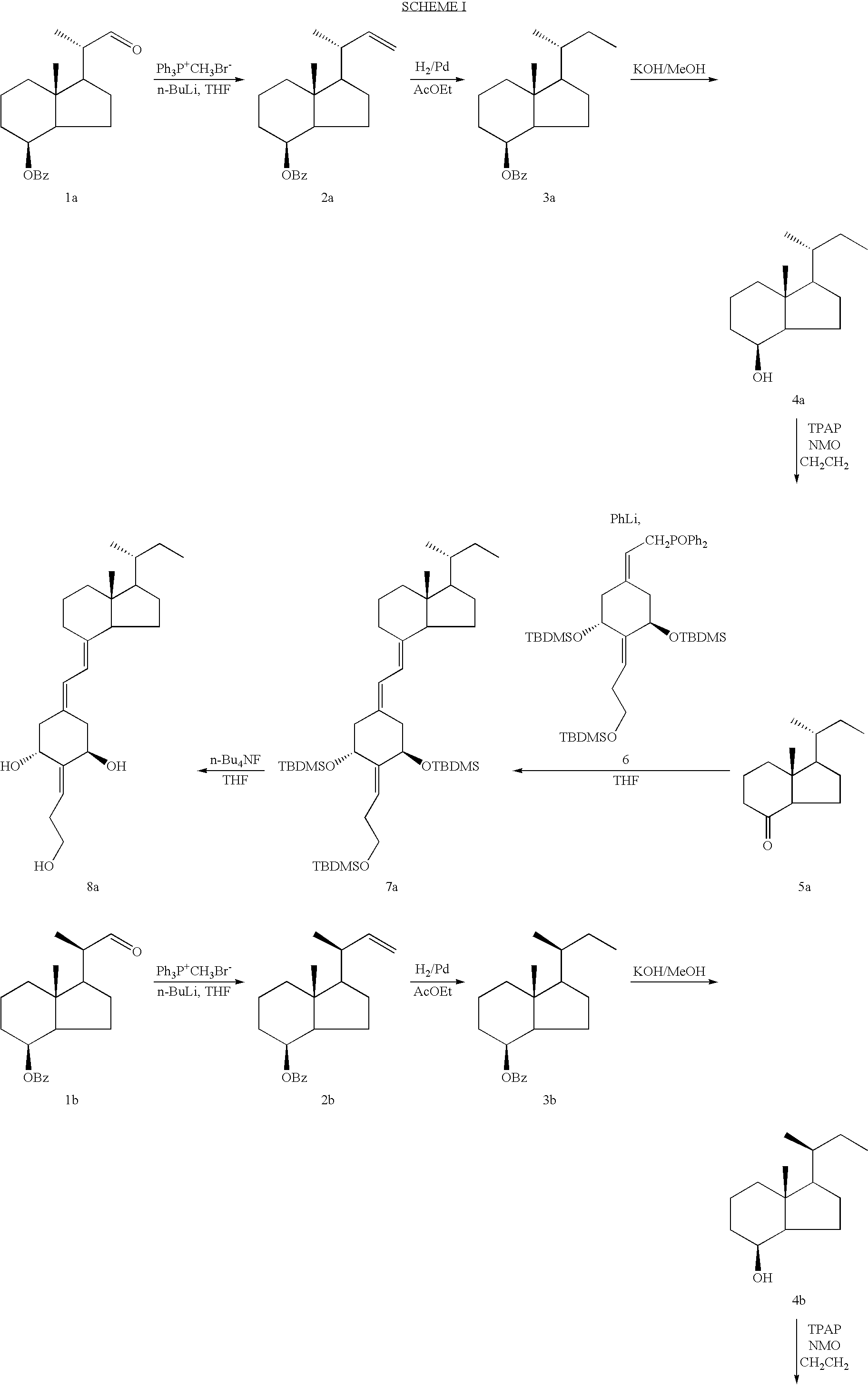

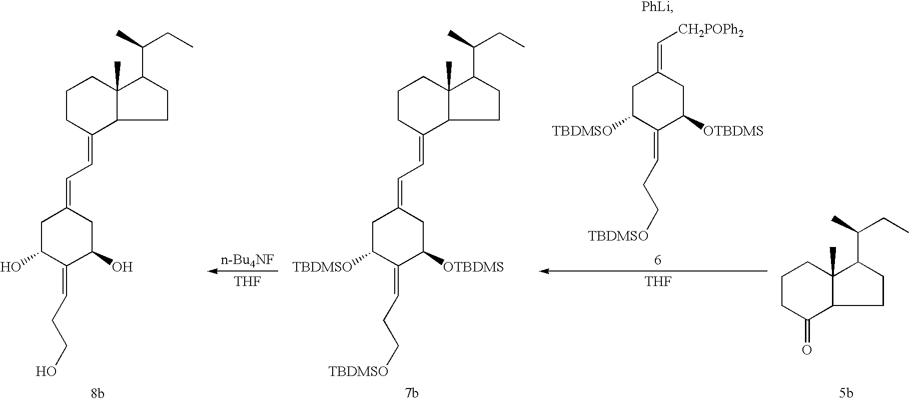

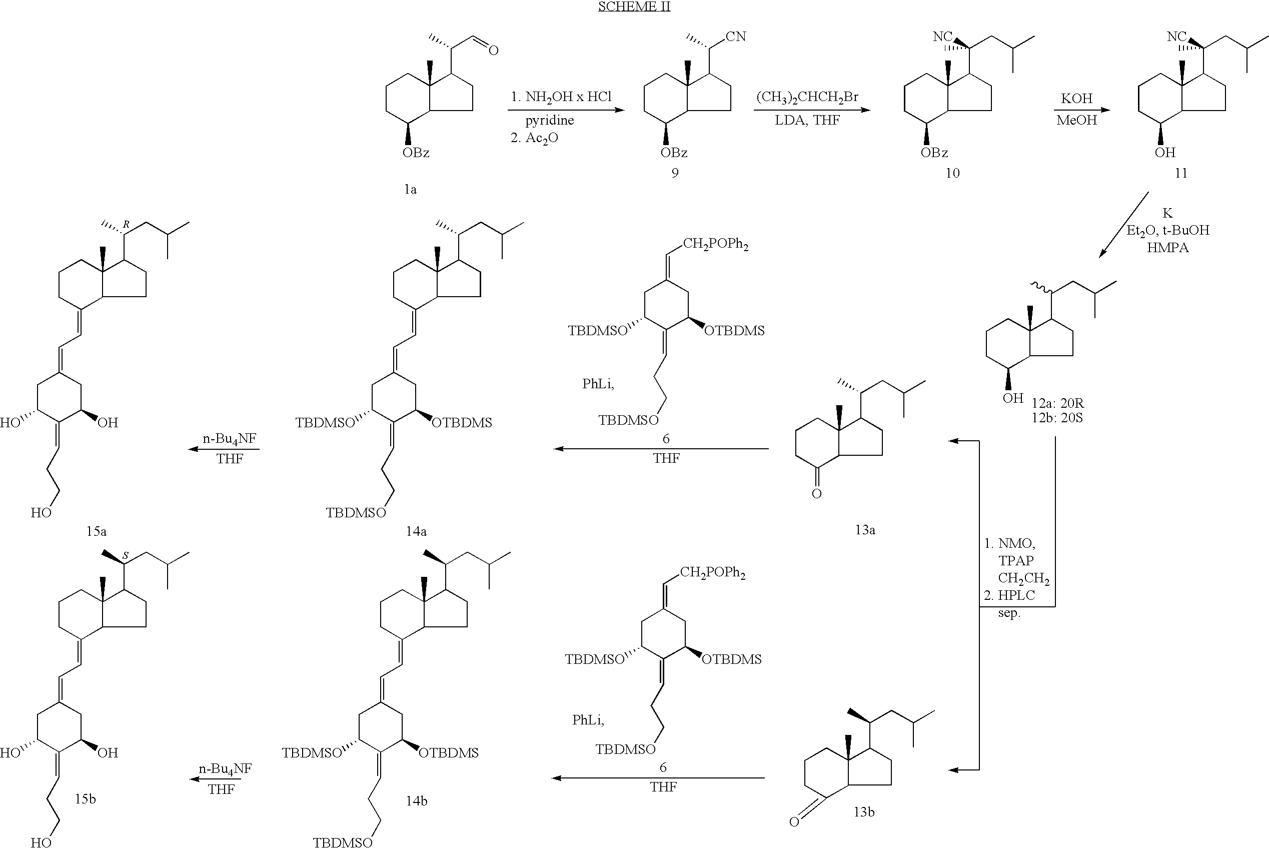

The term “OBZ” means a benzoate group according to the structure

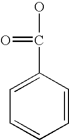

We claim:

1. A compound of the formula comprising: 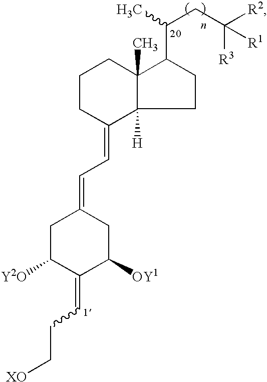
wherein [symbol] at C(1′) provides that the compound is an E- or Z-geometrical isomer respecting the 2-propylidene segment,
wherein the C(20) is the stereochemical center,
wherein the [symbol] at C20 is [symbol] or [symbol] providing an R or S configuration,
wherein n is an integer from 1 to 3,
wherein Y¹ is hydrogen, deuterium or a first hydroxy-protecting group,
wherein Y² is hydrogen, deuterium or a second hydroxy-protecting group,
wherein X is a third hydroxy-protecting group,
wherein R¹ is hydrogen, deuterium or methyl,
wherein R² is hydrogen, deuterium or methyl, and,
wherein R³ is hydrogen, deuterium or methyl,
and, hydrates thereof.

2. The compound of claim 1, wherein X is hydrogen, deuterium, C₁₋₁₀ branched or straight alkyl, C₁₋₁₀ branched or straight alkyl substituted with one or more hydroxy groups, C₁₋₁₀ branched or straight alkyl substituted with one or more C₁₋₁₀ branched or straight alkoxy groups, C₁₋₁₀ branched or straight alkyl substituted with one or more aryloxy groups, carbonyl substituted with one or more C₁₋₁₀ branched or straight alkoxy groups, C₁₋₆ branched or straight alkanoyl, C₁₋₆ branched or straight carboxyalkanoyl, aromatic acyl, silyl substituted with one or more C₁₋₁₀ branched or straight alkyl groups, silyl substituted with one or more C₁₋₁₀ branched or straight alkyl groups, or, silyl substituted with one or more aryl groups.

3. The compound of claim 2, wherein the carbonyl substituted with a C₁₋₁₀ branched or straight alkoxy groups is methoxycarbonyl, ethoxycarbonyl, propoxycarbonyl, iso-propoxycarbonyl, butoxycarbonyl, isobutoxycarbonyl, tert-butoxycarbonyl, benzyloxycarbonyl, or, allyloxycarbonyl.

4. The compound of claim 2, wherein the C₁₋₆ branched or straight carboxyalkanoyl is oxalyl, malonyl, succinyl, or, glutaryl, and, wherein the aromatic acyl is benzoyl, halo-substituted benzoyl, nitro-substituted benzoyl, or, C₁₋₁₀ straight or branched alkyl-substituted benzoyl.

5. The compound of claim 2, wherein the C₁₋₁₀ branched or straight alkyl substituted with one or more C₁₋₁₀ branched or straight alkoxy groups is methoxymethyl, ethoxymethyl, methoxyethoxymethyl, tetrahydrofuranyl or tetrahydropyranyl.

6. The compound of claim 2, wherein the silyl substituted with one or more C₁₋₁₀ branched or straight alkyl groups is trimethylsilyl, triethylsilyl, t-butyldimethylsilyl, or, dibutylmethylsilyl, and, wherein the silyl substituted with one or more aryl groups is diphenylmethylsilyl, phenyldimethylsilyl, or, diphenyl-t-butylsilyl.

7. The compound of claim 2, wherein the C₁₋₁₀ branched or straight alkyl substituted with one or more aryloxy groups is phenyl-substituted phenyl, C₁₋₁₀ straight or branched alkyl-substituted phenyl, nitro-substituted phenyl, or, halo-substituted phenyl.

8. The compound of claim 1, wherein the compound is an E-geometrical isomer.

9. The compound of claim 1, wherein the compound is a Z-geometrical isomer.

10. The compound of claim 6, wherein X is t-butyldimethylsilyl.

11. The compound of claim 10, wherein Y¹ is t-butyldimethylsilyl.

12. The compound of claim 11 wherein Y² is t-butyldimethylsilyl.

13. The compound of claim 1, wherein X is hydrogen.

14. The compound of claim 13, wherein Y¹ is hydrogen.

15. The compound of claim 14, wherein Y² is hydrogen.

16. The compound of claim 1, wherein n is 1, wherein R¹ and R² are methyl, and, wherein R³ is hydrogen.

17. A compound comprising an E-isomer of (20R)-1α-hydroxy-2-(3′-hydroxypropylidene)-19,24,25,26,27-penta-nor-vitamin D₃.

18. A compound comprising an E-isomer of (20S)-1α-hydroxy-2-(3′-hydroxypropylidene)-19,24,25,26,27-penta-nor-vitamin D₃.

19. A compound comprising an E-isomer of (20R)-1α-hydroxy-2-(3′-hydroxypropylidene)-19,23,24-tri-nor-vitamin D₃.

20. A compound comprising an E-isomer of (20S)-1α-hydroxy-2-(3′-hydroxypropylidene)-19,23,24-tri-nor-vitamin D₃.

21. A method of making a hydrindanone intermediate compound for use in making the compound of claim 1, wherein n is 1, wherein R¹, R², and R³ are each hydrogen, and, wherein [symbol] at C20 is [symbol] comprising:
providing a starting compound of the formula: 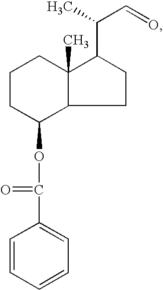
reacting the starting compound with a ylide reactant to produce an alkene-containing product,
hydrogenating the alkene-containing product to produce an oily ester product,
hydrolysing the oily ester product to produce an alcohol product, and,
oxidizing the alcohol product to produce the hydrindanone intermediate compound having the formula:

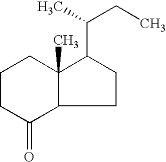

22. A method of making the compound of claim 1, wherein n is 1, wherein R¹, R², and R³ are each hydrogen, and, wherein [symbol] at C20 is [symbol] comprising:
coupling the hydrindanone intermediate compound of claim 21 with lithium phosphinoxy carbanion to produce a coupled product having the protecting groups, and,
hydrolyzing the protecting groups.

23. A method of making a hydrindanone intermediate compound for use in making the compound of claim 1, wherein n is 1, wherein R¹, R², and R³ are each hydrogen, and, wherein [symbol] at C20 is [symbol] comprising:
providing a starting compound of the formula: 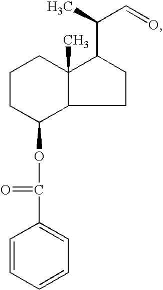
reacting the starting compound with a ylide reactant to produce an alkene-containing product,
hydrogenating the alkene-containing product to produce an oily ester product,
hydrolysing the oily ester product to produce an alcohol product, and,
oxidizing the alcohol product to produce the hydrindanone intermediate compound having the formula:

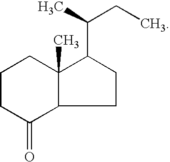

24. A method of snaking the compound of claim 1, wherein n is 1, wherein R¹, R², and R³ are each hydrogen, and, wherein [symbol] at C20 is [symbol] comprising:
coupling the hydrindanone intermediate compound of claim 21 with lithium phosphinoxy carbanion to produce a coupled product having the protecting groups, and,
hydrolyzing the protecting groups.

25. A method of making the compound of claim 1, wherein at least one of R¹, R² or R³ is a methyl, and, wherein [symbol] at C20 is [symbol] comprising:
providing a starting compound of the formula: 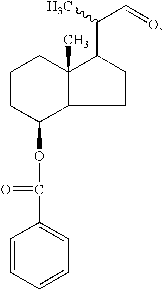
wherein [symbol] is [symbol] or [symbol]
converting the starting compound into a nitrile compound,
alkylating the nitrile compound with a first reactant of the formula: 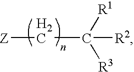
wherein n is an integer from 1 to 3, wherein Z is Br, Cl or I, and, wherein at least one of R¹, R² or R³ is a methyl to produce an alkylated nitrile product,
hydrolysing the alkylated nitrile product to produce a hydroxy nitrile product,
reductively decyanating the hydroxy nitrile product to produce a mixture of epimeric alcohol products,
oxidizing the mixture of epimeric alcohol products to produce a mixture of a 20S-ketone product and a 20R-ketone product,
separating the 20S-ketone and 20R-ketone products,
coupling the 20R-ketone product with lithium phosphinoxy carbanion to produce a coupled 20R product having the protecting groups, and,
hydrolysing the protecting groups.

26. The method of claim 25, wherein n is 1, wherein Z is Br, wherein R¹ and R² are methyl, and, wherein R³ is hydrogen.

27. A method of making the compound of claim 1, wherein at least one of R¹, R² or R³ is a methyl, and, wherein [symbol] at C20 is [symbol] comprising:
providing a starting compound of the formula: 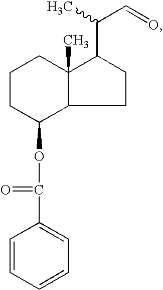
wherein [symbol] is [symbol] or [symbol]
converting the starting compound into a nitrile compound,
alkylating the nitrile compound with a first reactant of the formula: 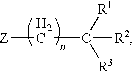
wherein n is an integer from 1 to 3, wherein Z is Br, Cl or I, and, wherein at least one of R¹, R² or R³ is a methyl, to produce an alkylated nitrile product,
hydrolysing the alkylated nitrile product to produce a hydroxy nitrile product,
reductively decyanating the hydroxy nitrile product to produce a mixture of epimeric alcohol products,
oxidizing the mixture of epimeric alcohol products to produce a mixture of a 20S-ketone product and a 20R-ketone product,
separating the 20S-ketone and 20R-ketone products,
coupling the 20S-ketone product with lithium phosphinoxy carbanion to produce a coupled 20S product having the protecting groups, and,
hydrolysing the protecting groups.

28. The method of claim 27, wherein n is 1, wherein Z is Br, wherein R¹ and R² are methyl, and, wherein R³ is hydrogen.
